(19) United States
(12) Patent Application Publication
(10) Pub. No.: US 2015/0291952 A1
(43) Pub. Date: Oct. 15, 2015

(54) RNA APTAMER ISOLATION VIA DUAL-CYCLE (RAPID) SELECTION

(71) Applicant: CORNELL UNIVERSITY, Ithaca, NY (US)

(72) Inventors: Harold G. Craighead, Ithaca, NY (US); David R. Latulippe, Paris (CA); John T. Lis, Ithaca, NY (US); Abdullah Ozer, Vestal, NY (US); Kylan Szeto, Ithaca, NY (US)

(73) Assignee: CORNELL UNIVERSITY, Ithaca, NY (US)

(21) Appl. No.: 14/421,720

(22) PCT Filed: Aug. 15, 2013
(86) PCT No.: PCT/US2013/055214

§ 371 Date: Feb. 13, 2015

Related U.S. Application Data
(60) Provisional application No. 61/683,381, filed on Aug. 15, 2012.

(51) Int. Cl. C12N 15/10; G06F 19/28; G06F 19/00; C12N 15/115

(52) U.S. Cl. CPC C12N 15/1058; C12N 15/115; G06F 19/28; G06F 19/702; C12N 2320/13

(57) ABSTRACT

The present invention relates to a method for selecting an aptamer for a target molecule. The method involves providing a random oligonucleotide library comprising a plurality of unique random sequence oligonucleotides; providing a target mixture comprising at least one target molecule; and subjecting the random oligonucleotide library and the target mixture to at least one round of an aptamer isolation protocol to yield at least one aptamer for the target molecule, wherein a round of the aptamer isolation protocol comprises at least one selection cycle followed by an amplification cycle. The present invention also relates to systems and devices for implementing or performing the method of the present invention. The present invention further relates to using the method to isolate aptamers for high-throughput sequencing analysis and other aptamer analysis protocols.

### CROSS-REFERENCE TO RELATED APPLICATIONS

This application claims priority benefit of U.S. Provisional Patent Application Ser. No. 61/683,381, filed Aug. 15, 2012, the disclosure of which is hereby incorporated by reference herein in its entirety.

### GOVERNMENT RIGHTS STATEMENT

This invention was made with Government support under grant numbers GM090320 and DA030329 awarded by the National Institutes of Health. The United States Government has certain rights in the invention.

### FIELD OF THE INVENTION

The present invention relates to a method for selecting an aptamer for a target molecule. The present invention also relates to protocols, methods, devices, and systems used in conjunction with the method for selecting an aptamer for a target molecule.

### BACKGROUND OF THE INVENTION

Aptamers are high-affinity ligands selected from large libraries of random oligonucleotides that can contain up to 10¹⁶ unique sequences. SELEX (Systematic Evolution of Ligands by EXponential enrichment) (Joyce 1989; Ellington and Szostak 1990; Tuerk and Gold 1990), an in vitro selection method, can isolate aptamers with high-affinity and specificity for a wide range of target molecules from an initial library of DNA or RNA sequences (Ciesiolka et al. 1995; Nitsche et al. 2007; Paige et al. 2011). This is achieved by iteratively selecting and amplifying target-bound sequences to preferentially enrich those sequences with the highest affinity to the target. Typically, after 10 to 15 iterations, one or several aptamers may be identified from the enriched pool, a process which can take months to complete. If an RNA aptamer is desired, this process takes longer due to additional steps required for reverse transcription into amplifiable cDNA and subsequent transcription back into RNA. A disproportionate amount of time and effort is dedicated to amplifying RNA pools compared to the actual selection steps where aptamer enrichment takes place. This not only adds significant time to the overall process, but also adds significant costs.

Recent work has focused on improving selection efficiency and on enriching for aptamers with particular target-binding properties. This has resulted in modifications to the conventional SELEX strategy including the use of multiple targets to control specificity (Jenison et al. 1994; Geiger et al. 1996; Gong et al. 2012), changing the characteristics of the random library (Latham et al. 1994; Green et al. 1995; Jensen et al. 1995; Klussmann et al. 1996; Ruckman et al. 1998; Burmeister et al. 2005; Gold et al. 2010), using different substrates for presentation of target molecules (Ellington and Szostak 1990; Daniels et al. 2003; Peng et al. 2007; Park et al. 2009; Cho et al. 2010), and varying the separation technique (Ellington and Szostak 1990; Mendonsa and Bowser 2004; Raddatz et al. 2008; Cho et al. 2010). Some work has been done to improve the throughput of aptamer discovery by utilizing high-throughput sequencing (Cho et al. 2010; Zimmermann et al. 2010; Schutze et al. 2011) or by performing parallel selections (Park et al. 2009; Jolma et al. 2010). A number of automated selection strategies have also been introduced (Cox et al. 1998). However, fully automated systems lack the routine quality controls and evaluations that are applied when manual selections are performed (Cox and Ellington 2001). Recently, a multiplexed microcolumn technique was reported that optimized selection parameters based on enrichment of a specific aptamer and demonstrated the ability to efficiently perform selections against multiple targets in parallel (Latulippe et al. 2013).

A major limiting step in many applications for aptamers is post-selection identification and refinement of candidates for diagnostic or therapeutic use. This is especially true when a high affinity aptamer also needs to be highly specific and to have a precise functional effect upon binding to its target, and effort may be put into characterizing, minimizing and modifying such aptamers. Unfortunately, these refinements are generally tested by trial and error, adding significant cost and time for aptamer discovery. A high-throughput assay for characterizing candidate aptamers for binding and a streamlined process for aptamer optimization is needed. However, these refinements ultimately depend on the initial quality of the aptamer selection, and there is still a lack of thorough characterization and knowledge about the most efficient or effective methods and conditions for performing selections with emerging technologies. Improvements in this domain would not only reduce the time and cost in performing selections, but have the potential to improve the rate and quality of downstream aptamer identification and refinement (Latulippe et al. 2013; Ozer et al. 2013)

Despite many advances, few selection approaches diverge from the core methodology of traditional SELEX. It is believed that only one technique breaks from the typical cycle of iterative and sequential selection and amplification steps; Non-SELEX (Berezovski et al. 2006) was shown to quickly generate DNA aptamers by repeated selections from an enriched library without any amplification steps. This methodology is useful for libraries that cannot be amplified. However, the capillary electrophoresis-based selection platform used for Non-SELEX requires tiny injection volumes (˜150 nL) to achieve efficient separations and only a small fraction of the sequences recovered from a given selection cycle are re-injected for the subsequent cycle. This constraint significantly lowers the total number of sequence candidates that can be investigated, and hence lowers the complexity and diversity of the injected library by 5 or 6 orders of magnitude. In addition, this method requires chemical modifications of the random library for fluorescence detection, which may alter its binding properties. Despite these restrictions, Non-SELEX was used to successfully identify DNA aptamers to h-RAS protein, bovine catalase and signal transduction proteins (Berezovski et al. 2006; Tok et al. 2010; Ashley et al. 2012), which suggests that in some cases aptamers may be much more abundant in random pools than previously thought. However, without amplification steps, this technique makes identifying aptamer candidates via population-based methods difficult. This limits the potential for using high-throughput sequencing, which has been used to characterize sequence distributions and their cycle-to-cycle dynamics, and has proven to be a powerful technique for identifying enriching aptamers with great sensitivity many cycles before true convergence (Cho et al. 2010; Schutze et al. 2011; Latulippe et al. 2013).

U.S. Pat. No. 5,792,613 to Schmidt et al. describes a method for obtaining RNA aptamers based on shape selection. The method purports to distinguish shape-recognizing RNA aptamers from RNA aptamers that bind the nucleic acid molecule primarily by way of base pairing interactions, such as Watson-Crick interactions. The disclosure does not teach a method of reducing the time and reagents needed to select RNA apatmers without compromising selection performance. Instead, as noted above, the disclosure is narrowly directed to selecting for an RNA aptamer based on binding of the aptamer to a structural element.

U.S. Pat. No. 8,314,052 to Jackson describes a method for simultaneous generation of functional ligands. The method is described as being useful for simultaneously generating numerous different functional biomolecules, particularly for generating numerous different functional nucleic acids against multiple target molecules simultaneously. However, the disclosure does not teach a method of reducing the time and reagents needed to select RNA apatmers without compromising selection performance.

Therefore, there is a need for a method for isolating aptamers in a robust and efficient way, particularly one that can yield accurate results while at the same time reducing the time and reagents needed to complete the aptamer selection.

The present invention is directed toward overcoming these and other deficiencies in the art.

### SUMMARY OF THE INVENTION

The present invention relates to a method for selecting an aptamer for a target molecule. The present invention also relates to protocols, methods, devices, and systems used in conjunction with the method for selecting an aptamer for a target molecule.

In one aspect, the present invention provides a method for selecting an aptamer for a target molecule. The disclosed method involves the following steps: providing a random oligonucleotide library comprising a plurality of unique random sequence oligonucleotides; providing a target mixture comprising at least one target molecule; and subjecting the random oligonucleotide library and the target mixture to at least one round of an aptamer isolation protocol to yield at least one aptamer for the target molecule.

According to this method, a round of the aptamer isolation protocol comprises at least one selection cycle followed by an amplification cycle. According to this method, the at least one selection cycle comprises: (i) contacting the random oligonucleotide library with the target mixture to bind oligonucleotides to the target molecule; and (ii) isolating the bound oligonucleotides to yield an enriched oligonucleotide pool comprising a plurality of high affinity oligonucleotides that bind with specificity to the target molecule. According to this method, the amplification cycle comprises subjecting the enriched oligonucleotide pool to an amplification process to yield an amplified oligonucleotide pool comprising an increased number of copies of the plurality of high affinity oligonucleotides.

In one embodiment, this method further comprises: determining that an amplification cycle trigger point has been reached before performing the amplification cycle. In this embodiment of the method, the amplification cycle trigger point is reached when either of the following occurs: (a) aptamer molecule numbers fall below a minimum acceptable number of molecules (Nₘᵢₙ); or (b) measured background binding probability approaches an assumed binding probability within a minimum acceptable enrichment factor (Eₘᵢₙ).

In another aspect, the present invention provides a method for selecting an aptamer for a target molecule that involves the following steps: providing a random oligonucleotide library comprising a plurality of unique random sequence oligonucleotides; providing a target mixture comprising at least one target molecule; and subjecting the random oligonucleotide library and the target mixture to multiple rounds of an aptamer isolation protocol to yield at least one aptamer that binds with specificity and high affinity to the target molecule.

According to this method, one round of an aptamer isolation protocol comprises multiple non-amplification selection cycles followed by one amplification cycle. The multiple non-amplification selection cycles initially comprises: (i) contacting the random oligonucleotide library with the target mixture to selectively bind a fraction of the oligonucleotide library to the target molecule; (ii) isolating the bound oligonucleotides to yield an enriched oligonucleotide pool; (iii) contacting the enriched oligonucleotide pool with the target mixture to selectively bind a fraction of the oligonucleotide pool to the target molecule; and (iv) repeating steps (ii) and (iii) to obtain an amount of the enriched oligonucleotide pool comprising a plurality of high affinity oligonucleotides, remaining for the amplification cycle. According this method, the amplification cycle comprises subjecting the enriched oligonucleotide pool to an amplification process to yield an amplified oligonucleotide pool comprising an increased number of copies of the plurality of high affinity oligonucleotides.

In one aspect, the present invention relates to a method for selecting an aptamer for a target molecule. The method involves providing a random oligonucleotide library comprising a plurality of unique random sequence oligonucleotides; providing a target mixture comprising at least one target molecule; and subjecting the random oligonucleotide library and the target mixture to at least one round of an aptamer isolation protocol to yield at least one aptamer for the target molecule, wherein a round of the aptamer isolation protocol comprises at least one selection cycle followed by an amplification cycle. The present invention also relates to systems and devices for implementing or performing the method of the present invention. The present invention further relates to using the method to isolate aptamers for high-throughput sequencing analysis and other aptamer analysis protocols.

In accordance with various aspects, the present disclosure provides a new method, RNA Aptamer Isolation via Dual-cycles (RAPID), that provides a generalized approach for accelerating the rate of aptamer selections. RAPID selections significantly decrease the cost and time needed for RNA aptamer selections by systematically eliminating unnecessary amplification steps and performing amplifications only when higher sequence copy numbers or higher pool concentrations are required. For each additional selection cycle performed without amplification (Non-Amplification Cycle), the additional cost and effort associated with RNA specific processing, such as reverse transcription and transcription reactions, are eliminated in addition to the typical DNA PCR amplification processes. This not only reduces the use of costly enzymes and reagents, but also minimizes the time required for aptamer selections. Furthermore, the RAPID method of the present invention can be applied to any selection mode and used with any technology, including those that utilize whole cells and target cell surface proteins as in Cell-SELEX (Daniels et al. 2003).

These and other objects, features, and advantages of this invention will become apparent from the following detailed description of the various aspects of the invention taken in conjunction with the accompanying drawings.

### BRIEF DESCRIPTION OF THE DRAWINGS

For the purpose of illustrating aspects of the present invention, there are depicted in the drawings certain embodiments of the invention. However, the invention is not limited to the precise arrangements and instrumentalities of the embodiments depicted in the drawings. Further, as provided, like reference numerals contained in the drawings are meant to identify similar or identical elements.

FIGS. 1A-1D illustrate one embodiment of an RNA Aptamer Isolation via Dual-cycles (RAPID) method of the present invention and results obtained by the method. FIG. 1A: Schematic diagram of one embodiment of the RAPID process of the present invention. The starting library or the enriched and amplified pool from the previous selection step can either go through the (inner) Non-Amplification Cycle and be used immediately in the next selection or go through the (outer) Amplification Cycle. FIG. 1B: An example of processing times for SELEX and RAPID to complete two full selection cycles. Each 3 hour selection step is indicated with black blocks and arrowheads (▾) on top. FIG. 1C: The total time required to complete six cycles of SELEX under optimal enrichment conditions, and six cycles of RAPID performed by alternating between Non-Amplification and Amplification Cycles; each coloured block represents the total processing time between amplification steps. Asterisks (*) indicate the enriched and amplified pools that were analysed via high-throughput sequencing. FIG. 1D: The total time required to complete six cycles of SELEX under optimal enrichment conditions, and six cycles of RAPID performed by alternating between Non-Amplification and Amplification Cycles; each coloured block represents the total processing time between amplification steps. Asterisks (*) indicate the enriched and amplified pools that were analysed via high-throughput sequencing.

FIGS. 2A-2C: Binding of RNA after each selection cycle. FIG. 2A: Percent RNA recovery for SELEX cycles for Empty microcolumns (orange circles), microcolumns filled with UBLCP1-loaded resin (red squares), and microcolumns filled with CHK2-loaded resin (blue triangles). In this mode, there is a clear distinction between the protein-bound and the Empty microcolumns. FIG. 2B: Percent RNA recovery for RAPID cycles for the same targets. In this mode, there are significant increases in the percent aptamer recoveries following selections with non-amplified pools at Cycles 2, 4, and 6, followed by a concentration induced drop with the amplified pools at Cycles 3 and 5. FIG. 2C: Test of enriched pool binding to CHK2 protein preparation. F-EMSA shows the progression of bulk binding affinity increase for both SELEX and RAPID enriched pools with the RAPID Cycle 6 pool showing higher bulk binding than the SELEX Cycle 6 pool.

FIGS. 3A-3C: Sequence multiplicity distributions for various cycles of SELEX and RAPID. FIG. 3A: Distributions of the top 10,000 highest multiplicity sequences for SELEX Cycles 3 to 6 for Empty, UBLCP1 and CHK2 targets. Multiplicity values have been normalized to counts per 10⁷. FIG. 3B: The same Sequence multiplicity distributions of RAPID Cycles 2, 4 and 6 for the same targets. FIG. 3C: The similarity between RAPID and SELEX pool distributions for each target as determined by calculating the percent overlap of each RAPID cycle's distribution with each SELEX cycle's for each sample. The highest valued SELEX cycle against a given RAPID cycle is considered to be most similar to the given RAPID cycle.

FIGS. 4A-4F: The relationship between sequence multiplicity and enrichment. FIGS. 4A and 4B: Scatter plots of sequences' multiplicity and enrichment within the top 10,000 highest multiplicity sequences from Cycle 6 of SELEX and RAPID for the Empty microcolumns. Multiplicity values have been normalized to counts per 10⁷ and enrichment is calculated as the ratio of Cycle 6 multiplicities to Cycle 4 multiplicities for any sequence found in both pools. Some data points are obscured due to overlapping values. FIGS. 4C and 4D: Scatter plots of sequences' multiplicity and Cycle 4-to-Cycle 6 enrichment within the top 10,000 highest multiplicity sequences from Cycle 6 of UBLCP1 SELEX and RAPID. FIGS. 4E and 4F: Scatter plots of sequences' multiplicity and enrichment within the top 10,000 highest multiplicity sequences from Cycle 6 of CHK2 SELEX and RAPID. RAPID sequences show significantly higher multiplicities at lower enrichments than SELEX.

FIGS. 5A-5D: Relationship of the SELEX and RAPID selected sequences in Cycle 6 pools. FIGS. 5A and 5B: The first 50 random bases of the top 5 highest multiplicity UBLCP1 sequences and CHK2 sequences from Cycle 6 in RAPID (top) and SELEX (bottom). Identical sequences between both methods are highlighted with matching colours. The ranks of each sequence at earlier cycles (4 and 5) are also shown. As shown in FIG. 5A (top), the UBLCP1 sequences from RAPID are, from top to bottom, as follows: SEQ ID NO:1, SEQ ID NO:2, SEQ ID NO:3, SEQ ID NO:4, and SEQ ID NO:5. As shown in FIG. 5A (bottom), the UBLCP1 sequences from SELEX are, from top to bottom, as follows: SEQ ID NO:6, SEQ ID NO:7, SEQ ID NO:8, SEQ ID NO:9, and SEQ ID NO:10. As shown in FIG. 5B (top), the CHK2 sequences from RAPID are, from top to bottom, as follows: SEQ ID NO:11, SEQ ID NO:12, SEQ ID NO:13, SEQ ID NO:14, and SEQ ID NO:15. As shown in FIG. 5B (bottom), the CHK2 sequences from SELEX are, from top to bottom, as follows: SEQ ID NO:16, SEQ ID NO:17, SEQ ID NO:18, SEQ ID NO:19, and SEQ ID NO:20. FIG. 5C: A scatter plot of the 687 common sequences for UBLCP1 in SELEX and RAPID Cycle 6 pools; the dashed line represents a 1:1 correlation between multiplicities in the two pools. FIG. 5D: The same analysis for CHK2 yielded 1317 common sequences. On average, RAPID pools were enriched above SELEX pools.

FIGS. 6A-6B: Binding test of the CHK2 protein prep's highest multiplicity Cycle 6 aptamer candidate C6M1. The sequence is given by the two flanking constant regions, and the random region: GATCGGTTCCAACGCTCTGTCGCCTAAGTGAAC AGATGAAGAAAAAATAGCCCAATAAGAGGCAACAATCT (SEQ ID NO:21). FIG. 6A: Gel image of F-EMSA for C6M1 aptamer incubated with no protein or the CHK2 protein prep ranging from 1.4 nM to 2000 nM, in 1.5-fold increments. FIG. 6B: Binding curves for C6M1 using F-EMSA and FP. The left axis shows the calculated fraction bound from F-EMSA (solid line, filled circles), while the right axis shows the fluorescence polarization from C6M1 (dotted line, empty circles); the fitted K_{d} for the two curves are 180±13 nM and 299±53 nM, respectively.

FIG. 7: Relationship of the sequence multiplicities for sequences that are common to both UBLCP1 and CHK2 selected RAPID Cycle 6 pools. Of the 2004 sequences of interest (687 and 1317 sequences common between Cycle 6 of RAPID and SELEX pools for UBLCP1 and CHK2, respectively), only 8 of them were also common between the two target pools. This is likely due to a trace cross-contamination and strongly suggests that the unique sequences in each pool are target specific.

FIG. 8: Fluorescent polarization binding assays of bulk SELEX pools to CHK2 prep. The fitted K_{d}'s for the Cycle 3 and Cycle 6 pools are both 1.6-fold higher than the corresponding F-EMSA results in FIG. 2C.

### DETAILED DESCRIPTION OF THE INVENTION

The present invention generally relates to, inter alia, methods, systems, and devices for selecting an aptamer for a target molecule. The present invention also relates to protocols, methods, devices, and systems used in conjunction with the method for selecting an aptamer for a target molecule.

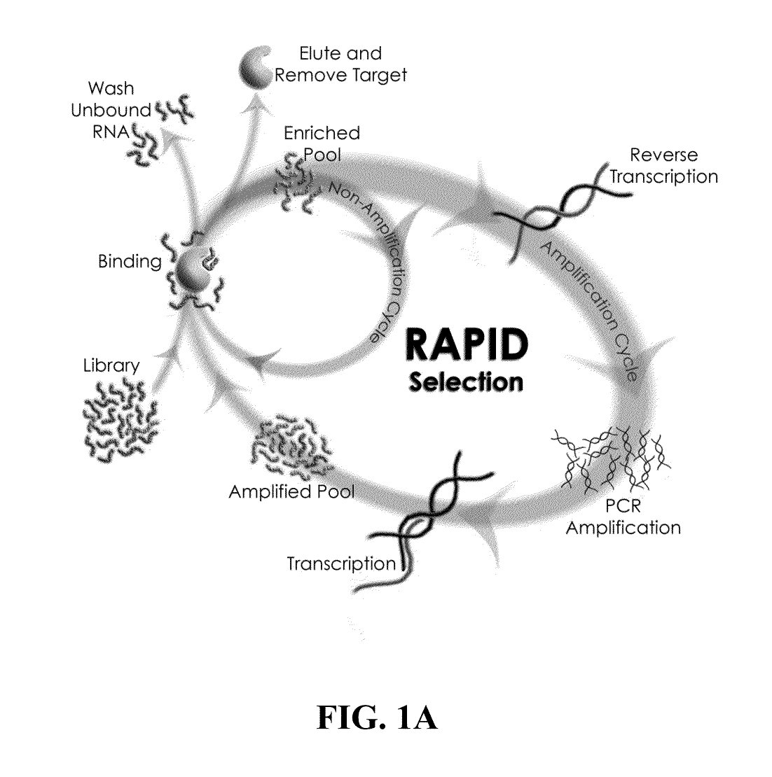

In one aspect, the present invention provides, inter alia, a new method for the efficient selection of RNA aptamers: RNA APtamer Isolation via Dual-cycle (“RAPID”) selection. A schematic representation of one embodiment of the RAPID method is illustrated in FIG. 1A. As shown in FIG. 1A, in one embodiment of the RAPID method, the starting RNA library is mixed with the target molecule and any unbound RNA are washed away. The target is then removed to yield an enriched pool of RNA molecules—this pool can then be subjected to one of two different cycles. If there is a sufficient quantity of RNA available, the enriched pool goes into the non-amplification cycle and is used directly in another selection step with the same target molecule. Alternatively, the enriched pool goes into the amplification cycle and thus gets reverse-transcribed into single-stranded DNA, amplified by PCR, and then transcribed back into RNA; the overall result is a new amplified pool that is then used in another selection step with the same target molecule. In this method, the total number of “rounds” is defined as the number of amplification cycles and so one round can include multiple non-amplification cycles.

This method is clearly different than any of the prior technologies in the relevant art and has the following new benefits:

First, it is considerably faster than the conventional SELEX method. FIG. 1B shows a time-line plot of the various steps in a typical RNA aptamer selection process with a 3-hour selection step. A single round of conventional SELEX requires approximately 24 hours of total experimental time; the majority of the time (˜80-90%) is spent on the amplification steps that are needed to prepare the new RNA pool for the next selection step. However, the RAPID selection with one non-amplification cycle and one amplification cycle requires only 28 hours or less. Therefore, the RAPID selection strategy would achieve the same number of selections and save at least 20 hours of experimental time for every two rounds of conventional SELEX.

Second, the RAPID selection strategy of the present disclosure is compatible with nearly any aptamer selection technique including nitrocellulose filter binding, affinity tags or surfaces, microfluidic devices, flow cytometry, surface plasmon resonance, and centrifugation (see review by Gopinath (Anal Bioanal Chem (2007) 387:171-182)).

Third, high-throughput sequencing techniques can be used to analyze the amplified pools from each amplification cycle (i.e., round) and determine which specific aptamers are being enriched in the successive rounds of selection. Multiple pools can be sequenced simultaneously (by use of a barcode sequence to distinguish them) to investigate the behavior of individual aptamers and confirm a lack of biases in either the pools or the processing.

Fourth, the starting library has the same complexity as conventional SELEX strategies (˜10¹⁵ different sequences).

Fifth, fewer amplification cycles for the same number of selections would require only a fraction of the enzymes and other biological materials required for each process in the amplification step.

In another aspect, the present invention provides a method for selecting an aptamer for a target molecule, where the aptamer is not limited to an RNA aptamer, but can also be a DNA aptamer. The disclosed method involves the following steps: providing a random oligonucleotide library comprising a plurality of unique random sequence oligonucleotides; providing a target mixture comprising at least one target molecule; and subjecting the random oligonucleotide library and the target mixture to at least one round of an aptamer isolation protocol to yield at least one aptamer for the target molecule.

According to this method, a round of the aptamer isolation protocol comprises at least one selection cycle followed by an amplification cycle. According to this method, the at least one selection cycle comprises: (i) contacting the random oligonucleotide library with the target mixture to bind oligonucleotides to the target molecule; and (ii) isolating the bound oligonucleotides to yield an enriched oligonucleotide pool comprising a plurality of high affinity oligonucleotides that bind with specificity to the target molecule. According to this method, the amplification cycle comprises subjecting the enriched oligonucleotide pool to an amplification process to yield an amplified oligonucleotide pool comprising an increased number of copies of the plurality of high affinity oligonucleotides.

In one embodiment, this method further comprises: determining that an amplification cycle trigger point has been reached before performing the amplification cycle. In this embodiment of the method, the amplification cycle trigger point is reached when either of the following occurs: (a) aptamer molecule numbers fall below a minimum acceptable number of molecules (Nₘᵢₙ); or (b) measured background binding probability approaches an assumed binding probability within a minimum acceptable enrichment factor (Eₘᵢₙ).

In determining when the amplification cycle trigger point is reached and when an amplification cycle should be performed, the present disclosure provides various means for making this determination, as set forth below.

In one embodiment of this method, the number of selection cycles (denoted as “i”) before an amplification cycle is to be performed is determined based on the minimum acceptable number of molecules (Nₘᵢₙ) as calculated according to Formula I as follows:

Nₘᵢₙ×P(A)≧N(A)×P(A)ⁱ (Formula I)

wherein:
Nₘᵢₙ×P(A)≧1;
Nₘᵢₙ≧N(A)×P(A)ⁱ⁻¹, where Nₘᵢₙ≧P(A)⁻¹≧1 and i≧1;
N(A)=number of such molecules believed to be present, where N(A)≧1;
P(A)=probability of binding an aptamer molecule; and
i=the number of selection cycles before an amplification cycle is to be performed, and
wherein the amplification cycle trigger point is reached and an amplification cycle is to be performed once the inequality of Formula I becomes untrue after “i” cycles.

In another embodiment of this method, the number of selection cycles (denoted as “i”) before an amplification cycle is to be performed is determined based on the minimum acceptable number of molecules (Nₘᵢₙ) as calculated according to Formula I as follows:

Nₘᵢₙ×P(A)≧N(A)×P(A)ⁱ (Formula I)

wherein:
Nₘᵢₙ×P(A)≧1;
Nₘᵢₙ≧N(A)×P(A)ⁱ⁻¹, where Nₘᵢₙ≧P(A)⁻¹≧1 and M>i>1;
M=total number of selection cycles to be performed;
N(A)=number of such molecules believed to be present, where N(A)≧1;
P(A)=probability of binding an aptamer molecule; and
i=the number of selection cycles before an amplification cycle is to be performed, and
wherein the amplification cycle trigger point is reached and an amplification cycle is to be performed once the inequality of Formula I becomes untrue after “i” cycles.

In a further embodiment of this method, the number of selection cycles (denoted as “i”) before an amplification cycle is to be performed is determined based on the minimum acceptable enrichment factor (Eₘᵢₙ) as calculated according to Formula II as follows:

Eₘᵢₙ≧P(A)/P(B,n,i) (Formula II)

wherein:
Eₘᵢₙ>1 and n≧i≧1;
P(A)=probability of binding an aptamer molecule;
P(B,n,i)=measured probability of binding background molecules at the n^{th} cycle with i cycles performed after the last amplification; and
i=the number of selection cycles before an amplification cycle is to be performed,
wherein the amplification cycle trigger point is reached and an amplification cycle is to be performed once the inequality of Formula II becomes untrue after “i” cycles.

In yet another embodiment of this method, the number of selection cycles (denoted as “i”) before an amplification cycle is to be performed is determined based on the minimum acceptable enrichment factor (Eₘᵢₙ) as calculated according to Formula II as follows:

Eₘᵢₙ≧P(A)/P(B,n,i) (Formula II)

wherein:
Eₘᵢₙ>1 and n≧i≧1 and M>i;
P(A)=probability of binding an aptamer molecule;
P(B,n,i)=measured probability of binding background molecules at the n^{th} cycle with i cycles performed after the last amplification; and
i=the number of selection cycles before an amplification cycle is to be performed,
wherein the amplification cycle trigger point is reached and an amplification cycle is to be performed once the inequality of Formula II becomes untrue after “i” cycles.

As set forth above and herein, in various embodiments, one round of the aptamer isolation protocol includes at least one selection cycle followed by an amplification cycle. The number of selection cycles in a particular round of the aptamer isolation protocol can include, without limitation, between one and ten selection cycles and in certain embodiments more than ten selection cycles, depending on the particular target molecule and/or random oligonucleotide library, as well as other parameters desired or set forth by one of ordinary skill in the relevant art. Therefore, in various embodiments of this method, the step of determining when the amplification cycle trigger point is reached and when an amplification cycle should be performed can be done, without limitation, after one selection cycle, after two selection cycles, after three selection cycles, after four selection cycles, after five selection cycles, after six selection cycles, after seven selection cycles, after eight selection cycles, after nine selection cycles, after ten selection cycles, and after more than ten selection cycles.

As set forth above and herein, a minimum acceptable number of aptamer molecules (Nₘᵢₙ) after a selection cycle can be used to trigger when an amplification cycle should be performed. In various embodiments, suitable Nₘᵢₙ values can include, without limitation, a value in a range selected from the group consisting of from between about 1 and about 500 aptamer molecules, between about 1 and about 400 aptamer molecules, between about 1 and about 300 aptamer molecules, between about 1 and about 200 aptamer molecules, between about 1 and about 100 aptamer molecules, between about 1 and about 90 aptamer molecules, between about 1 and about 80 aptamer molecules, between about 1 and about 70 aptamer molecules, between about 1 and about 60 aptamer molecules, between about 1 and about 50 aptamer molecules, between about 1 and about 40 aptamer molecules, between about 1 and about 30 aptamer molecules, between about 1 and about 20 aptamer molecules, between about 1 and about 15 aptamer molecules, between about 1 and about 10 aptamer molecules, and between about 1 and about 5 aptamer molecules.

As set forth above and herein, a minimum acceptable enrichment factor (Eₘᵢₙ) after a selection cycle can be used to trigger when an amplification cycle should be performed. As discussed herein, Eₘᵢₙ is used in relation to background binding, where a trigger for performing an amplification cycle is when the measured background probability approaches an assumed binding probability within a minimum acceptable enrichment factor (Eₘᵢₙ). In various embodiments, suitable Eₘᵢₙ values can include, without limitation, a value in a range selected from the group consisting of within about 1/1000 of probability of binding an aptamer molecule (denoted as “P(A)”), within about 1/500 of P(A), within about 1/400 of P(A), within about 1/300 of P(A), within about 1/200 of P(A), within about 1/100 of P(A), within about 1/50 of P(A), within about 1/25 of P(A), within about 1/20 of P(A), within about 1/15 of P(A), within about 1/10 of P(A), within about ⅕ of P(A), within about 1 of P(A), and within about 10 of P(A).

Therefore, as described above and herein, in accordance with various embodiments of the method of the present invention, the number of selection cycles to be performed in a particular round of the aptamer isolation protocol is dependent on reaching an amplification cycle trigger point, wherein the amplification cycle trigger point is reached when either of the following occurs: (a) aptamer molecule numbers fall below a minimum acceptable number of molecules (Nₘᵢₙ); or (b) measured background binding probability approaches an assumed binding probability within a minimum acceptable enrichment factor (Eₘᵢₙ).

In accordance with various embodiments of the method of the present invention, the random oligonucleotide library and the target mixture can be subjected to various numbers of round of the aptamer isolation protocol. In particular embodiments, the random oligonucleotide library and the target mixture are subjected to one round, two rounds, three rounds, or more than three rounds of the aptamer isolation protocol. Thus, in other embodiments, the present invention can involve four, five, six, seven, eight, nine, ten, and more than ten rounds of the aptamer isolation protocol, as can be decided by one of ordinary skill in the relevant art.

In accordance with various embodiments of the method of the present invention, one round of the aptamer isolation protocol can include, without limitation, one selection cycle followed by one amplification cycle, two selection cycles followed by one amplification cycle, three selection cycles followed by one amplification cycle, four selection cycles followed by one amplification cycle, and more than four selection cycles followed by one amplification cycle. Thus, in other embodiments, the present invention can involve five, six, seven, eight, nine, ten, and more than ten selection cycles followed by one amplification cycle, as can be decided by one of ordinary skill in the relevant art.

In various embodiments of the method of the present invention, when more than one round of the aptamer isolation protocol is performed, each such round can have the same or different number of selection cycles before an amplification cycle is performed, as can be determined by one of ordinary skill in the relevant art.

In accordance with various embodiments of the method of the present invention, the trigger to perform an amplification cycle following a selection cycle can be based on the concentration of oligonucleotides left in the enriched oligonucleotide pool after a given selection cycle. In various particular embodiments, the amplification cycle is performed once there is, without limitation, about <0.10 pico-mols of oligonucleotides, about <0.05 pico-mols of oligonucleotides, about <0.04 pico-mols of oligonucleotides, about <0.03 pico-mols of oligonucleotides, about <0.02 pico-mols of oligonucleotides, or about <0.01 pico-mols of oligonucleotides left in the enriched oligonucleotide pool.

In accordance with various embodiments, the method of the present invention can be used for isolating aptamers for target molecules from any oligonucleotide library as understood by those of ordinary skill in the relevant art. In a particular embodiment, the oligonucleotide library is a random oligonucleotide library. More particularly, the random oligonucleotide library can be a random RNA oligonucleotide library or a random DNA oligonucleotide library.

In accordance with various embodiments, the method of the present invention can be used for isolating various types of aptamers for various types of target molecules, as understood by those of ordinary skill in the relevant art.

In a particular embodiment, the aptamer is selected from the group consisting of an RNA aptamer and a DNA aptamer. In other embodiments, the aptamer can be a mixture of different RNA aptamers, different DNA aptamers, or a mixture of both RNA and DNA aptamers.

In particular embodiments, the target molecule can include, without limitation, a whole cell, a virus, a protein, a modified protein, a polypeptide, a modified polypeptide, an RNA molecule, a DNA molecule, a modified DNA molecule, a polysaccharide, an amino acid, an antibiotic, a pharmaceutical agent, an organic non-pharmaceutical agent, a macromolecular complex, a carbohydrate, a small molecule, a chemical compound, a mixture of lysed cells, and a mixture of purified, partially purified, or non-purified protein.

The methods of the present invention can be used in conjunction with any protocol, method, system, or device that relates to the isolation, purification, analysis, sequencing, amplification, and use of aptamers, whether the aptamers are RNA aptamers or DNA aptamers. Depending on the desired use of the method of the present invention, those of ordinary skill can readily understand how the presently disclosed method for selecting an aptamer for a target molecule can be used in conjunction with any such protocol, method, system, or device. The present invention also relates to the combined protocols, methods, systems, or devices as combined with the method of the present disclosure.

In certain particular embodiments of the method of the present invention, the step of isolating the bound oligonucleotides to yield the enriched oligonucleotide pool comprises: washing unbound and weakly bound oligonucleotides from the target mixture (e.g., as used with a microcolumn device or system); and eluting the oligonucleotides that specifically bind to the target molecules, wherein the eluted oligonucleotides are aptamers that bind to the target molecules.

In one embodiment, when the oligonucleotide aptamers comprise RNA aptamers, the method can further comprise performing reverse transcription amplification of the selected aptamer population. In other embodiments, the method can still further comprise purifying and sequencing the amplified apatmer population.

In one embodiment of the method of the present invention, the performing reverse transcription amplification, the purifying, and/or the sequencing are performed in one or more separate fluidic devices coupled in fluidic communication with a microcolumn device suitable for maintaining a target molecule.

In another aspect, the present invention provides a method for selecting an aptamer for a target molecule that involves the following steps: providing a random oligonucleotide library comprising a plurality of unique random sequence oligonucleotides; providing a target mixture comprising at least one target molecule; and subjecting the random oligonucleotide library and the target mixture to multiple rounds of an aptamer isolation protocol to yield at least one aptamer that binds with specificity and high affinity to the target molecule.

According to this method, one round of an aptamer isolation protocol comprises multiple non-amplification selection cycles followed by one amplification cycle. The multiple non-amplification selection cycles initially comprises: (i) contacting the random oligonucleotide library with the target mixture to selectively bind a fraction of the oligonucleotide library to the target molecule; (ii) isolating the bound oligonucleotides to yield an enriched oligonucleotide pool; (iii) contacting the enriched oligonucleotide pool with the target mixture to selectively bind a fraction of the oligonucleotide pool to the target molecule; and (iv) repeating steps (ii) and (iii) to obtain an amount of the enriched oligonucleotide pool comprising a plurality of high affinity oligonucleotides, remaining for the amplification cycle. According to this method, the amplification cycle comprises subjecting the enriched oligonucleotide pool to an amplification process to yield an amplified oligonucleotide pool comprising an increased number of copies of the plurality of high affinity oligonucleotides.

As with the first method described herein, this method can include various embodiments, some of which are described herein below, but are not meant to be limiting of this method.

In one embodiment of this method, the multiple non-amplification selection cycles comprises two selection cycles, three selection cycles, or more than three selection cycles.

In one embodiment of this method, the random oligonucleotide library and the target mixture are subjected to two rounds, three rounds, or more than three rounds of the aptamer isolation protocol.

In one embodiment of this method, one round of the aptamer isolation protocol is selected from the group consisting of two selection cycles followed by one amplification cycle, three selection cycles followed by one amplification cycle, four selection cycles followed by one amplification cycle, and more than four selection cycles followed by one amplification cycle.

In one embodiment of this method, the amplification cycle is performed once there is about <0.10 pico-mols of oligonucleotides, about <0.05 pico-mols of oligonucleotides, about <0.04 pico-mols of oligonucleotides, about <0.03 pico-mols of oligonucleotides, about <0.02 pico-mols of oligonucleotides, or about <0.01 pico-mols of oligonucleotides left in the enriched oligonucleotide pool.

In one embodiment of this method, the random oligonucleotide library is a random RNA oligonucleotide library or a random DNA oligonucleotide library.

In one embodiment of this method, the aptamer is selected from the group consisting of an RNA aptamer and a DNA aptamer.

In one embodiment of this method, the target molecule is selected from the group consisting of a whole cell, a virus, a protein, a modified protein, a polypeptide, a modified polypeptide, an RNA molecule, a DNA molecule, a modified DNA molecule, a polysaccharide, an amino acid, an antibiotic, a pharmaceutical agent, an organic non-pharmaceutical agent, a macromolecular complex, a carbohydrate, a small molecule, a chemical compound, a mixture of lysed cells, and a mixture of purified, partially purified, or non-purified protein.

### EXAMPLES

The following examples are intended to illustrate particular embodiments of the present invention, but are by no means intended to limit the scope of the present invention.

### Example 1

### RAPID Selection of RNA Aptamers

Aptamers are high-affinity ligands selected from random DNA or RNA libraries via SELEX, a repetitive in vitro process of sequential selection and amplification steps. Compared to DNA however, RNA SELEX is complicated and lengthened by the additional amplification steps of transcription and reverse transcription. Here, we report a new selection method, RAPID (RNA Aptamer Isolation via Dual-cycles), that simplifies this process by systematically skipping unnecessary amplification steps. RAPID provides a generalized approach that can be used with any selection technology to accelerate the rate of aptamer discovery. Using affinity microcolumns, we were able to complete a multiplex selection against two protein targets, CHK2 and UBLCP1, in less than half the time required for analogous selections using the conventional SELEX approach. High-throughput sequencing of the enriched pools from both SELEX and RAPID revealed many identical candidate aptamers from the starting pool of 5×10¹⁵ sequences. For CHK2, the same sequence was preferentially enriched in both selections as the top candidate and was found to bind to its respective target. These results demonstrate the efficiency and, most importantly, the robustness of our selection schemes. RAPID, therefore, reduces the time and reagents needed to select RNA aptamers, without compromising selection performance.

Here, we demonstrate the improved efficiency of RAPID, by comparing and analyzing its sequence candidates to those generated from conventional SELEX using our previously-described, microcolumn-based platform (Latulippe et al. 2013) to the target proteins, CHK2 and UBLCP1. After completing six selection cycles, RAPID had enriched the same candidates on average 3-fold more and at half the cost and requiring only a third of the time as SELEX.

### Materials and Methods

### Protein Preparation

As previously described (Latulippe et al. 2013), recombinant hexahistidine-tagged CHK2 and UBLCP1 proteins were expressed in BL21(DE3)-RIPL E. coli cells (Agilent Technologies). LB cultures supplemented with 100 μg/ml ampicillin were inoculated with starter LB culture derived from a single colony and grown at 37° C. until OD₆₀₀ reached 0.6. Protein expression was induced with 0.2 mM IPTG at 18-22° C. for ˜16 hours. After centrifugation, the bacterial pellet was collected and processed according to the manufacturer's instructions for Ni-NTA Superflow resin (Qiagen). SDS-PAGE was used to determine the purity and quality of the final protein product. The resulting proteins were dialyzed with 1×PBS with 5 mM 2-mercaptoethanol and 0.01% Triton X-100. The proteins were evaluated for purity (˜90-95%) and were stored in small aliquots with 20% glycerol.

### RNA Library Preparation

As previously described (Latulippe et al. 2013), a synthesized DNA library was purchased from GenScript. To increase the diversity of the initial library and to include higher order RNA structural classes, we chose to use a random region of 70 nucleotides (nt); this length averages about 4.5 structural features (vertexes) (Gevertz et al. 2005). Including flanking constant regions, sequences in the library have 120 nts, as described by the scheme: 5′-AAGCTTCGTCAAGTCTGCAGTGAA-N70-GAATTCGTAGATGTGGATCCA TTCCC-3′ (SEQ ID NO:22). This length is the practical limit for efficient commercial synthesis of DNA templates. The single-stranded DNA template library was converted to double-stranded DNA while introducing the T7 promoter using Klenow exo-(NEB) and the Lib-FOR oligonucleotide, 5′-GATAATACGACTCACTATAGGGAATGGATCCACATC TACGA-3′ (SEQ ID NO:23). The resulting library was later amplified in a 1 L PCR reaction using Taq DNA polymerase, Lib-FOR oligonucleotide, and the Lib-REV oligo, 5′-AAGCTTCGTCAAGTCTGCAGTGAA-3′ (SEQ ID NO:24). A single aliquot capturing the complexity of the entire library (5×10¹⁵ unique sequences) was transcribed with T7 RNA polymerase in an 88 mL reaction yielding 1200-fold amplification. An aliquot of this RNA library, corresponding to an average of 4 to 6 copies of each unique sequence, was used as the starting pool for each selection method.

### Multiplex SELEX and RAPID

The protein immobilization was described previously (Latulippe et al. 2013). Briefly, a new batch of resin was prepared for each protein target. Ni-NTA Superflow resin was incubated in binding buffer (25 mM Tris-HCl, pH 8.0, 10 mM NaCl, 25 mM KCl, 5 mM MgCl₂) with each protein to the optimal final concentration of ˜0.6 μg protein/μl of resin and then loaded into custom fabricated microcolumns (Latulippe et al. 2013). For both SELEX and RAPID, three microcolumns were serially connected beginning with an Empty microcolumn, followed by UBLCP1 and ending with CHK2. Fresh aliquots of the RNA Library were prepared in 1 mL binding buffer by heat denaturing at 65° C. for 5 minutes, renaturing at 25° C. for 30 minutes and finally adding 200 U of Superase-In RNase Inhibitor (Invitrogen). 10 μL samples were taken as 1% standards for subsequent quantitation by qPCR.

For the SELEX cycles, 1 mL of blocking buffer (binding buffer supplemented with 0.3 μg/μL yeast tRNA) was injected into the microcolumn assembly at a rate of 100 μL/min to block any non-specific binding sites. We previously showed that a 1 μL/min flow rate for the library binding step yielded the highest enrichments (Latulippe et al. 2013), so the library was injected at this optimum rate using a multi-rack syringe pump (Harvard Apparatus). After binding the library, the microcolumns were reconfigured to run in parallel, and a 3 mL washing step was performed with binding buffer containing 10 mM imidazole. Similarly, we used a wash flow rate of 3 mL/min, which was shown to maximize enrichments over the starting library. Finally, bound sequences were collected from the microcolumns by flowing 400 μl of elution buffer (binding buffer supplemented with 50 mM EDTA) at 50 μL/min. Each RNA sample was then phenol:chloroform and chloroform extracted, ethanol precipitated together with 1 μl of GlycoBlue (Ambion) and 40 μg of yeast tRNA (Invitrogen), and re-suspended in 20 μl of DEPC-treated water. These were then reverse transcribed, PCR amplified, and transcribed into RNA (see below for details) for the next selection cycle. Five more SELEX cycles using the three microcolumns were completed in parallel, decreasing the washing flow rate by 10-fold at Cycles 3 and 6 to accommodate possible increases in the bulk affinity of the enriched pools. The input material was also decreased by 20-fold each cycle from Cycle 2 to 4 to decrease the time and reagents needed.

For the RAPID cycles, 1 mL of blocking buffer was injected into the serial microcolumn assembly at 100 μL/min. The library injections were performed at 10 μL/min to allow the completion of multiple selection cycles in one day. For the wash step, we used a 3 mL two-step “hybrid” wash at 1 mL/min for 1 minute, followed by 70 μL/min for 29 minutes. This combined the observed benefits of a brief, harsh wash for eliminating weakly bound or unbound molecules, with that of a longer wash for discriminating among more strongly bound molecules (Latulippe et al. 2013). In addition, this format eliminated the need to tune the washing flow rate as the cycles progressed, as was done for the SELEX cycles. Elution buffer was then injected to recover bound sequences, which were then phenol:chloroform and chloroform extracted, ethanol precipitated, re-suspended in 1 mL binding buffer, and then used as the input pool for the next selection cycle. We took 1% standards/samples from each new pool and then the selection steps were repeated with all of the microcolumns in parallel. Following the completion of the elution step after the second cycle, each RNA sample was extracted, precipitated, and re-suspended in 20 μL of DEPC-treated water. These were then reverse transcribed, PCR amplified, and transcribed into RNA (see below for details) for the next selection cycle. Two more RAPID “dual-cycles” (one Non-Amplification and one Amplification Cycle) were completed using the three microcolumns in parallel, decreasing the input material by 20-fold after each amplification cycle (Cycle 3 and 5).

The amplification and quantification of both the SELEX and RAPID pools were performed in the same way. All the resuspended samples and standards were reverse transcribed in 60 μL reactions with MMLV-RT and 30 pmol of Lib-REV primer. The cDNA samples were treated with RNaseH (Ambion) to eliminate the RNA and a small amount analysed on a LightCycler 480 qPCR instrument (Roche) to determine the amount of RNA that was retained on each microcolumn after each cycle and to determine the optimal number of PCR cycles needed to fully amplify each pool. 400 μL PCR reactions with 300 pmol of each primer were performed for each pool, followed by phenol:chloroform and chloroform extractions, and finally purified using DNA Clean & Concentrator (Zymo Research) spin columns. A small fraction (˜¼) of the purified PCR product was used to generate new RNA pools in 72 μL transcription reactions with T7 RNA polymerase. The template DNA was removed by DNaseI digestion and the resulting RNA pool was purified by phenol:chloroform and chloroform extractions and ethanol precipitation.

### High-Throughput Sequencing and Analysis

A detailed description has been reported (Latulippe et al. 2013). Briefly, PCR products from each target pool for various selection rounds were PCR amplified using 6 nt barcoded primers with adapters for the HiSeq 2000 (Illumina) sequencing platform. The barcoded PCR products were PAGE-purified, phenol:chloroform and chloroform extracted, ethanol precipitated, and then re-suspended in 10 mM Tris-HCl pH 7.5 buffer. High-throughput sequencing was performed by the sequencing core facility at Life Sciences Core Laboratories Center, Cornell University. After removing ambiguous and poor scoring sequences the remaining sequences were separated into pools based on the barcode sequences. Then sequences with 85% sequence identity were clustered together. This identity threshold is set to ensure that truly unique sequences with 85% identity (or higher) are unlikely to be present even within our large library size (2.5×10¹⁵) due to the vast potential 70 nt random sequence space (4⁷⁰=˜1.4 10⁴²) and thus such detected sequences account for PCR and sequencing errors. The sequence with the highest number of reads, hereafter referred to as the sequence multiplicity, within each cluster was identified as the cluster's true sequence and used as the representative sequence for that cluster. The total multiplicity of a cluster was defined as the sum of multiplicities within the cluster. All the representative sequences in each pool were sorted based on their multiplicity to identify candidate aptamers for each protein target. Sequence comparisons, histograms and scatterplots were performed and generated in MATLAB (Mathworks).

### Candidate Sequence Purification

The DNA templates for candidate aptamers were PCR amplified from the final Cycle 6 pool using Phusion Polymerase (NEB), the Lib-REV oligonucleotide, and an aptamer-specific oligonucleotide that spans the forward constant region and approximately 30 nt of the candidate's unique, random region. The resulting PCR product was double-digested with BamHI and PstI, and ligated using low melt agarose “in-gel” ligation (EZ Clone Systems) into a similarly cut pGEM3Z-N70Apt plasmid. PGEM3Z-N70Apt plasmid was obtained by cloning a random full-length aptamer template from the N70 library together with T7 promoter into the pGEM3Z vector (Promega) between Nan and HindIII sites. Three clones were sequenced to obtain a consensus for the full-length sequence of each candidate aptamer. The RNA aptamer was transcribed from the candidate's DNA templates, which were generated by PCR from the sequenced plasmid using the same primers.

### Fluorescence EMSA and Polarization Assays

The RNA samples were 3′-end labelled with fluorescein 5-thiosemicarbazide (Invitrogen) as described previously (Pagano et al. 2007). 50 μL binding reactions were prepared with 2 nM fluorescently-labelled RNA and decreasing amounts of protein (2000 to 0 nM) in binding buffer containing 0.01% IGEPAL CA630, 10 μg/ml yeast tRNA, and 3 U of SUPERase•In RNase Inhibitor. Reactions were prepared in black 96-well half area microplates (Corning) and incubated at room temperature for 2 hours. The plates were scanned on a Synergy H1 microplate reader (BioTek) using the Ex: 485/20 Em: 528/20 filter set to determine the Fluorescence Polarization (FP). The polarization “P” is determined from the total parallel and perpendicular polarized fluorescence according to:

$$P = \frac{{F}_{∥} - {F}_{⊥}}{{F}_{∥} + {F}_{⊥}} .$$

For Fluorescence Electrophoretic Mobility Shift Assays (F-EMSA), the same samples used for the FP measurements were spiked with 6× loading dye and loaded into the wells of a refrigerated 5% agarose gel prepared with 0.5×TBE buffer. The gel was run for 90 minutes at 120 volts in refrigerated 0.5×TBE buffer. Images were acquired using the fluorescein scan settings on a Typhoon 9400 imager (GE Healthcare Life Sciences) and the resulting bands were quantified with ImageJ. The dissociation constant, K_{d}, was determined by fitting the results from the FP and F-EMSA to the Hill equation:

$$Y = {Y}_{0} + \frac{{Y}_{MAX} - {Y}_{0}}{1 + {[ \frac{{K}_{d}}{X} ]}^{n}} .$$

### Results

### SELEX Versus RAPID

Traditional SELEX is performed by taking a random library and binding, partitioning, and amplifying target-bound sequences until an aptamer emerges. To achieve this, SELEX requires selections to be done by iterating sequentially through these steps. To improve the efficiency and reduce the cost of performing these selections, we developed a new method that incorporates a secondary cycle that does not include amplification. For simplicity, we differentiate these as Amplification and Non-Amplification Cycles (FIG. 1A). In doing so, RNA selections can be performed in much less time, and require less reagents and other costly materials. Example timelines for two cycles of SELEX and of RAPID selections performed under identical conditions are shown in FIG. 1B. Completion of one cycle of SELEX takes about 28 hours, over 80% of which is needed for the amplification step. In contrast, by skipping one Amplification Cycle, the RAPID method completes two selection cycles in nearly the same amount of time. These improvements could be even greater for configurations where multiple Non-Amplification Cycles are performed in between two subsequent Amplification Cycles. For both methods, we define a “round” of selection to necessarily include amplifications steps. In this way, a round of RAPID is comparable to a round of SELEX in both time and cost (a round and a cycle remain interchangeable terms in SELEX).

To properly evaluate the advantage of using RAPID, we completed six selection cycles using both methods. SELEX took a total of 255 hours to complete the six cycles of selections in six rounds using the previously determined optimal parameters for aptamer enrichment on the microcolumns (Latulippe et al. 2013). RAPID took 84 hours to complete six cycles in three rounds using parameters below optimal in order to complete each round's binding steps within one day, where each round comprised of a single Non-Amplification Cycle followed by an Amplification Cycle (FIG. 1C). With this design, RAPID took one third the time to complete as SELEX. In addition to reducing time and cost, removing unnecessary amplification steps minimizes its potential bias (Zimmermann et al. 2010; Thiel et al. 2011) and also reduces input libraries and pools to more convenient size scales when performing amplifications. Thus, rapid sequence convergence can be obtained by optimizing the number of Non-Amplification Cycles, while diverse sequence populations with high aptamer copy numbers are maintained through critical periodic Amplification Cycles.

### Ensemble Binding of Enriched Aptamer Pools

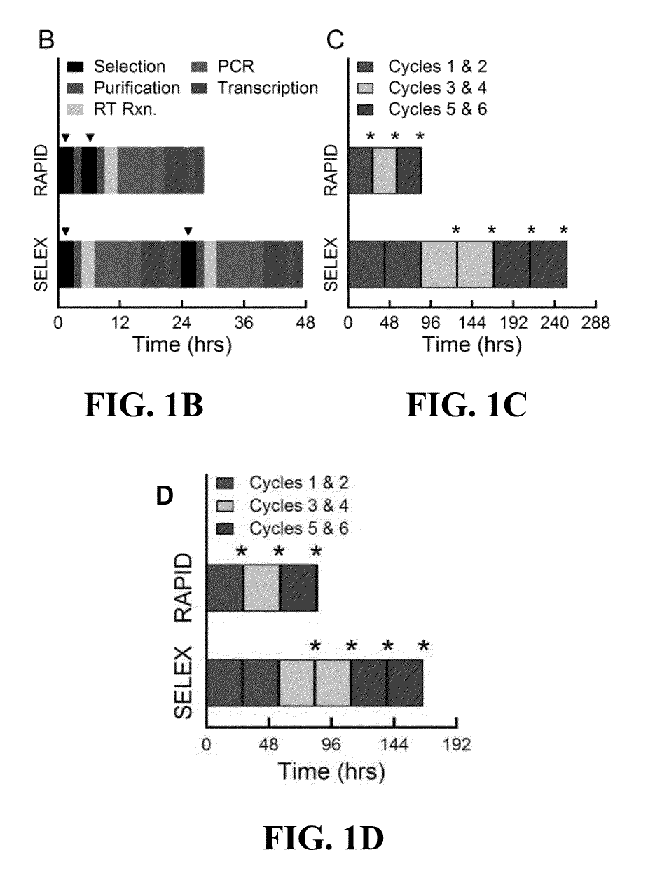

In order to monitor the progress of the selections, the recovery of bound RNA at each cycle was measured using quantitative PCR (qPCR). FIG. 2A shows the binding results for all six cycles of SELEX to the Empty, UBLCP1 and CHK2 microcolumns. A generally smooth and sigmoidal increase in binding for all three samples can be observed from cycle to cycle. However the two protein targets show better binding than the Empty microcolumn, with the CHK2 target demonstrating the most improved binding across the SELEX cycles. FIG. 2B shows the binding results for all six cycles of RAPID to each of the three targets. Compared to SELEX, retention of the RAPID pools showed fluctuations (˜0.01% of Input material in Cycle 1 vs. 0.1 to 1% in Cycle 2) characteristic of the varying input concentrations from cycle to cycle. This is expected since the input material from a Non-Amplification pool is lower in concentration causing increased binding as seen in Cycles 2, 4, and 6. Similarly, amplification of input RNA results in higher concentrations and an attendant decreased binding in Cycles 3 and 5. Despite these concentration induced fluctuations, CHK2 still consistently showed the higher binding of the two protein targets.

To evaluate the progress of the selections, we performed Fluorescence Electrophoretic Mobility Shift Assays (F-EMSA) for the initial random library and several enriched pools of the CHK2 protein target (RAPID Cycles 2, 4 and 6; SELEX Cycles 3 and 6). We report the dissociation constant, K_{d}, with the uncertainty of the fit, as well as the percent of input RNA that was bound at the highest protein concentration. The input library had a K_{d} value greater than 1 μM, with 59% of input RNA bound. The results shown in FIG. 2C indicate a general improvement in bulk affinity and an increased pool binding fraction at later cycles. For SELEX, the Cycle 3 pool had a K_{d}=315±26 nM (69% bound) while the Cycle 6 pool had K_{d}=281±24 nM (86% bound). For RAPID, the Cycle 2, 4, and 6 pools had K_{d} values of 390±34 nM (65% bound), 209±19 nM (72% bound), and 191±7 nM (87% bound), respectively. Across the cycles, the fraction of bound RNA increased monotonically from 59% for the starting library to 87% for the RAPID cycle 6 pool. In addition, the RAPID Cycle 6 pool showed a higher bulk affinity for the protein than the SELEX Cycle 6 pool, which suggests that RAPID was performing better than SELEX at enriching the pools.

Population Distributions from High-Throughput Sequencing Analysis of Selection Pools

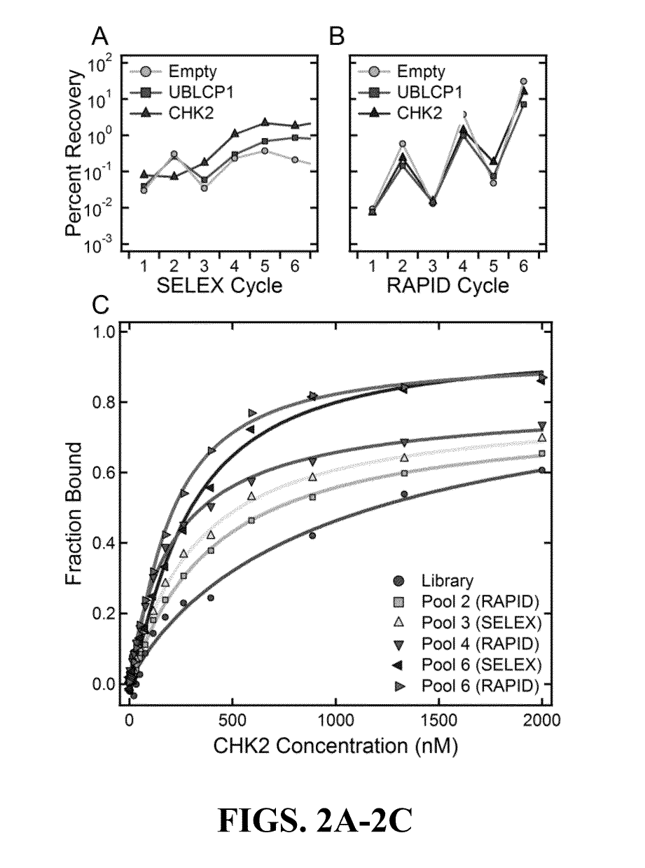

To identify candidate aptamers, we performed high-throughput sequencing on the selected pools. This also allowed us to rigorously compare the cycle-to-cycle enrichments of specific sequences from both the SELEX and RAPID selection methods as well as identify common sequences enriched by SELEX and RAPID (discussed below). We sequenced the four SELEX pools for Cycles 3, 4, 5, and 6 and all three of the amplified RAPID pools, Cycles 2, 4 and 6 (as indicated in FIG. 1C). Each pool had a different number of total sequencing reads (ranging from 5.6 to 9.4 million reads), so to compare values from different pools, the multiplicities were normalized to 10⁷ reads. We chose to analyse the top 10,000 highest multiplicity sequences from each pool because this was sufficient to cover 10-20% of the total sequence reads from the Cycle 6 pools, and also to simplify the analysis. The top 10,000 sequences for each pool were plotted as a histogram to compare the population distributions for each of the SELEX and RAPID pools in FIGS. 3A and 3B, respectively. The histograms clearly show for both methods the evolution of the protein targets' pool distributions toward higher multiplicities at higher cycle numbers. As expected, there was minimal increase in multiplicity observed in the Empty columns. This is consistent with the notion that RNA molecules bind randomly and non-specifically to the Empty column, without enriching any specific RNA sequence.

In order to make a more quantitative comparison of the evolution progress between the SELEX and RAPID distributions, we calculated the similarity between both methods' distributions. This is shown in FIG. 3C for each target by determining the percent overlap between each RAPID cycle distribution with each SELEX cycle distribution. We find that the RAPID pools for both protein targets were all further evolved than the SELEX pools. For example, the RAPID Cycle 2 and 4 distributions were most similar to the “later” SELEX Cycle 3 and 5 distributions, respectively. In addition, the RAPID Cycle 6 pool was more evolved than the SELEX Cycle 6 pool, though these two pools show maximal overlap since we only performed 6 cycles of selections for each method. For the Empty columns, we find that the overlap values are close to 100% between all of the pools confirming that there was negligible evolution within the Empty column's pools.

### Cycle 4-Cycle 6 Sequence Enrichments

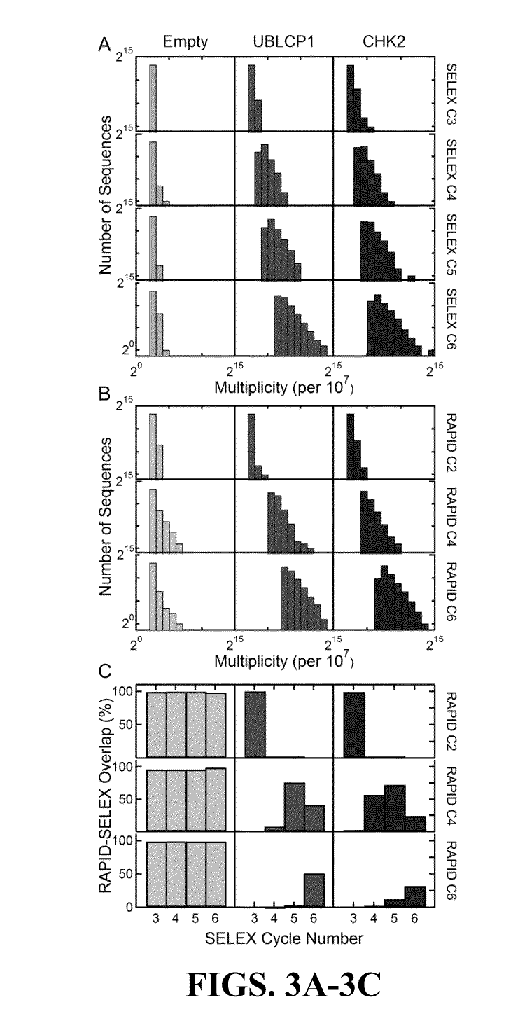

To further investigate and compare the evolution of pools between RAPID and SELEX cycles, we looked specifically at the enrichment of individual sequences. Enrichment was calculated from the ratio of multiplicity values from different cycles (Cho et al. 2010). The multiplicity values for the top 10,000 sequences in Cycle 6 were plotted versus their corresponding enrichment values from Cycle 4 to Cycle 6 for both selection methods (FIG. 4). For both protein targets, these two metrics were well correlated. However, we found that compared to the SELEX pools (FIGS. 4C and 4E), the RAPID pools (FIGS. 4D and 4F) have even higher multiplicities at equivalent enrichments. This suggests that the RAPID pools had enriched faster in earlier cycles. Although it is difficult to discern visually the number of individual data points in each panel, close examination of the data in these plots also show that for the protein targets, many of the top sequences were identified in both Cycle 6 and Cycle 4. Specifically, in the SELEX pools, UBLCP1 and CHK2 had 3,281 and 3,262 sequences, respectively, ranking in the top 10,000 of both pools. In the RAPID pools, UBLCP1 and CHK2 had 6,565 and 5,063 sequences, respectively, in common between the Cycle 4 and 6 pools' top 10,000 sequences. These results clearly show that RAPID pools have almost twice as many preserved sequences between cycles over SELEX, which is consistent with the improved evolution and enrichment data. In contrast, FIGS. 4A and 4B show that the Empty column had very few sequences ranking in the top 10,000 of both pools with 4 in SELEX and 8 in RAPID. In addition, the majority of those sequences had enrichment values less than one between the two cycles, which is expected if the binding and copy number for those sequences is random.

### Common Sequences Between SELEX and RAPID Cycle 6

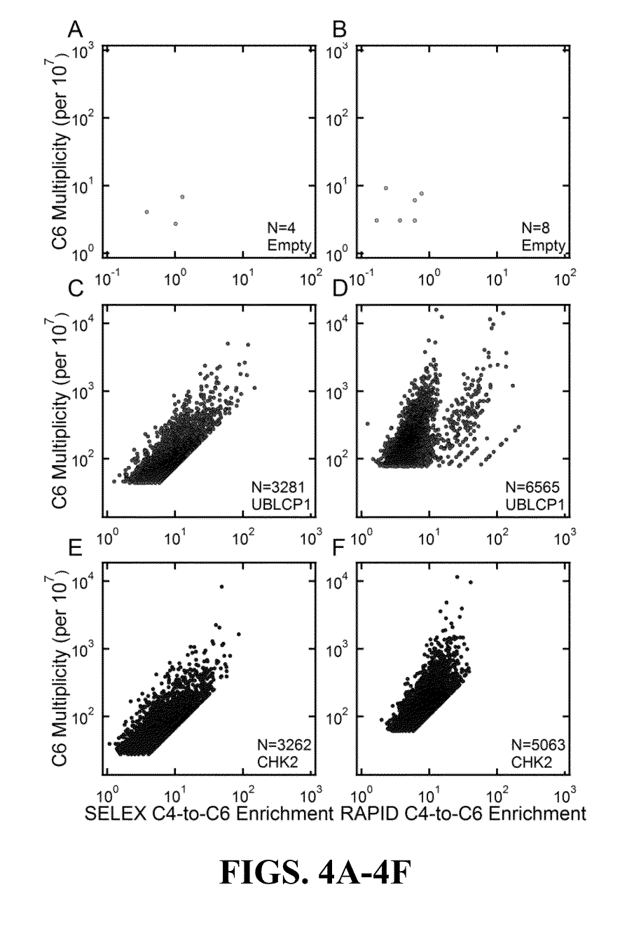

To determine the robustness of our two selection schemes, we looked more closely at a few of the top sequence candidates for the two Cycle 6 pools for each protein target. We found that among the top 5 ranked candidates, UBLCP1's top-ranked sequence in RAPID was ranked fifth in SELEX and its top-ranked sequence in SELEX was ranked third in RAPID (FIG. 5A). In addition, the top-ranked sequence in RAPID Cycle 6 was already top ranked in Cycle 4 and ranked second in SELEX Cycle 4. Similarly, the top-ranked sequence in SELEX Cycle 6 was ranked third in Cycle 4, first in Cycle 5 and second in RAPID Cycle 4. Furthermore, the top-ranked CHK2 sequence in RAPID was also ranked first in SELEX and was already ranked first in SELEX Cycles 4 and 5 as well as in RAPID Cycle 4 (FIG. 5B).

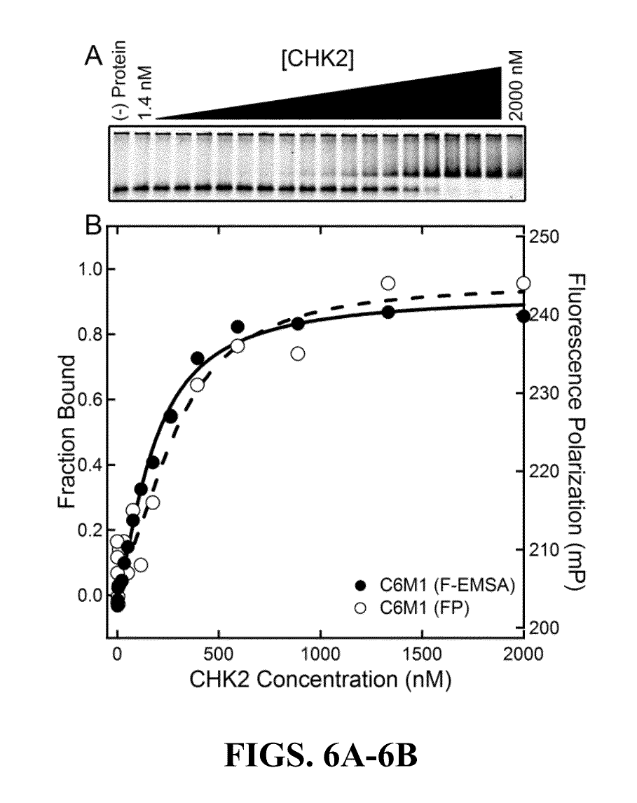

To extend this analysis, we searched for additional sequences common to each target's RAPID and SELEX Cycle 6 pools and found that many sequences among their top 10,000 were common and highly represented in both methods. Scatter plots relating the multiplicities of sequences represented in both pools are shown in FIGS. 5C and 5D. In total, we found 687 sequences that were common in both UBLCP1 pools, and 1317 sequences that were common in both CHK2 pools. Analysis for the Empty column yielded only a single common sequence with negligible multiplicities. Almost all of the common sequences were unique to each target (FIG. 7) and most appeared more highly enriched in the RAPID Cycle 6 pools. On average, the RAPID selected sequences represented higher fractions of their pools having enriched approximately 3-fold more than from SELEX: UBLCP1 by a factor of 2.6*2.3 (1.1-6.0-fold) and CHK2 by a factor of 2.8*2.2 (1.3-6.2-fold). These were determined by finding the geometric mean and standard deviation for the enrichments, thus the enrichments and their standard deviations are expressed as multiplicative factors.

### Aptamer Binding to CHK2 Protein

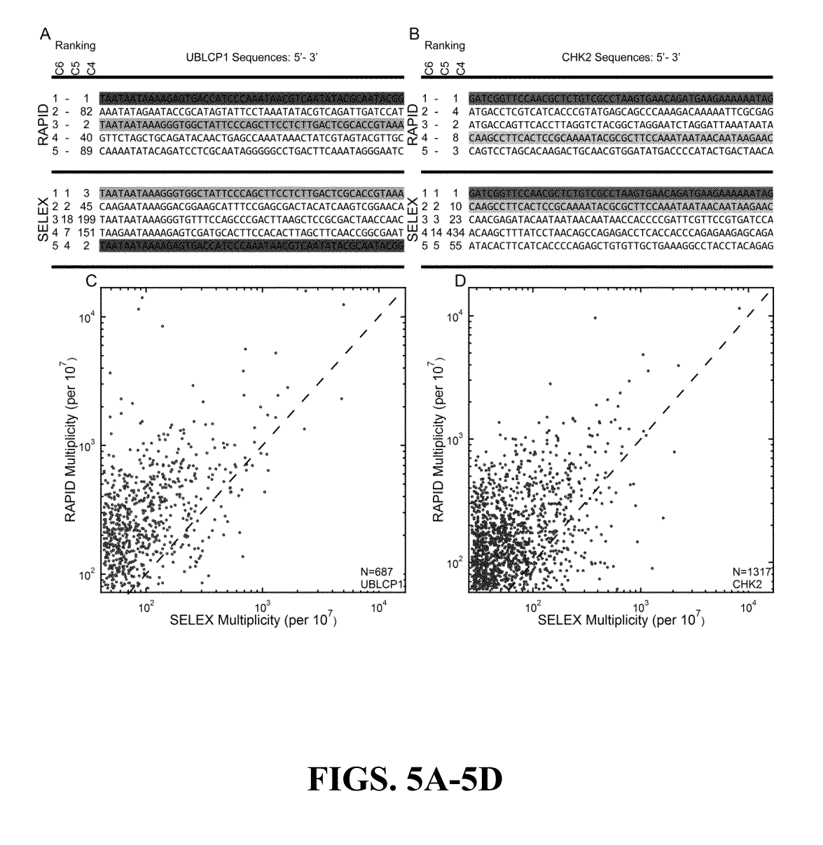

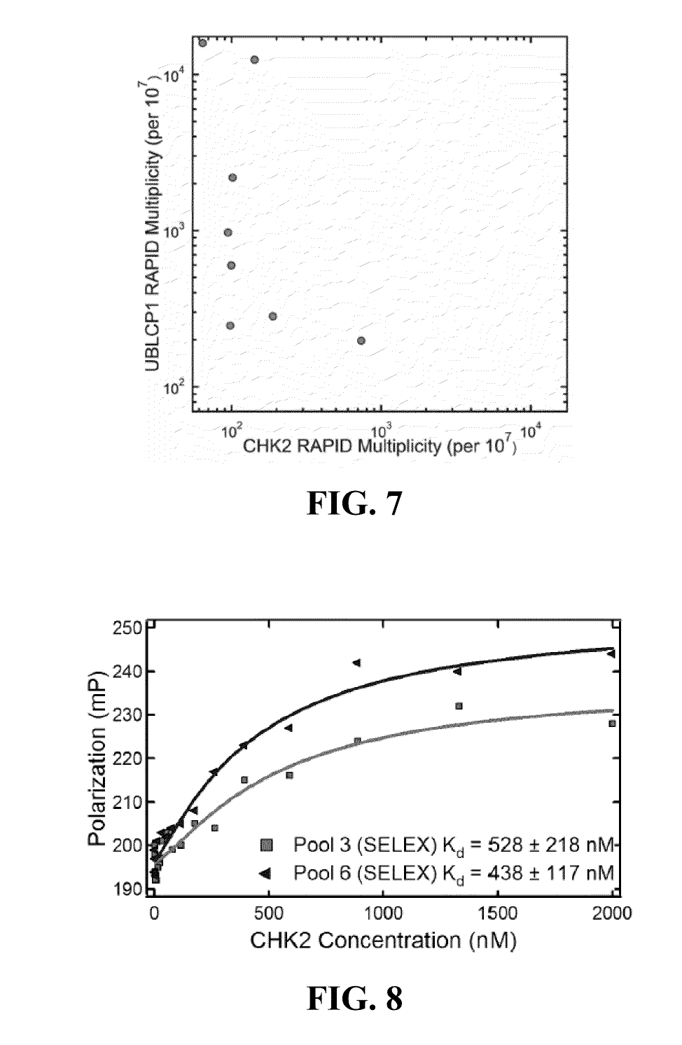

In order to confirm that the two methods had independently enriched the same aptamer, we tested the unambiguous top-ranked SELEX/RAPID candidate for CHK2 binding, hereafter referred to as C6M1. After isolating C6M1 from the Cycle 6 pools, we labelled the aptamer with fluorescein and performed Fluorescent Electromobility Shift Assay (F-EMSA) to assess its binding affinity to the same CHK2 protein preparation used for selections. FIG. 6A shows an image of the resulting gel shift assay. The calculated fraction bound curve for the gel shift is also shown in FIG. 6B as the solid line (left axis). These data were fit to the Hill equation and yielded a K_{d} of 180±13 nM. In order to ensure that the observed binding was not a gel artefact, we also performed a Fluorescence Polarization (FP) assay. The polarization curve is also shown in FIG. 6B as the dotted line (right axis). The calculated K_{d} is 299±53 nM, which is 1.6-fold higher than determined with F-EMSA. This factor is consistent with other FP assays performed on some of the labelled bulk SELEX pools (FIG. 8). Currently, we have not ruled out potential aptamer binding to a contaminant in our protein preparation. If this were the case, given the purity of our preparations, we would likely have underestimated the binding affinity by at least an order of magnitude and thus the aptamer would have a K_{d}<20 nM. However, for the purposes of this example, the results and conclusions of this work remain the same in either case.

### DISCUSSION

The RAPID selection method presented here is capable of isolating aptamers in less time and using fewer reagents than the conventional SELEX method. Standard binding assays with the amplified pools clearly revealed cycle-to-cycle affinity enrichment for two protein targets, CHK2 and UBLCP1, using both RAPID and conventional SELEX. Further, higher affinities and total binding to CHK2 were observed for pools from later selection cycles. We found that, of the two Cycle 6 pools, the RAPID pool had a higher affinity (˜1.5-fold higher) and could be bound at a higher fraction than the SELEX pool. This suggests that even though the RAPID selections were not performed with the optimal flow conditions used in SELEX, the lower input concentrations of the Non-Amplified RAPID pools benefited the overall selection, which would support the use of our RAPID method in many if not most selection strategies.

These qualitative trends continue when looking more closely at the individual sequences within each pool. As with the binding affinities, we found that despite having half the amplification steps as SELEX, the RAPID pools generated comparable if not better distribution profiles. This is in good agreement with the nicely ordered binding curves for the various pools mentioned above. In fact, these same binding results predicted that the RAPID pools would have slightly more evolved distributions, which is exactly what we observed (FIG. 3). Recalling our definition of a selection “round” that necessarily includes amplification steps, we found that one RAPID round was most similar to three SELEX rounds in terms of performance. Similarly, two RAPID rounds yielded performance similar to five SELEX rounds. This is particularly noteworthy since we found that our top candidate aptamers had acquired their high rankings after just two RAPID rounds (four cycles). The higher evolution of the RAPID pools is also supported by the higher slopes from the RAPID multiplicity versus enrichment scatter plots of sequences between the Cycle 4 and Cycle 6 pools.

Finally, we found that among the top ranked sequences from both methods, a large percentage (7% and 13%) were identical. This observation of independently enriched sequences demonstrates the effectiveness of both of our selection methods. However, in further support of the RAPID method, we found that among those identical sequences, the great majority were more enriched, an average of ˜3-fold, in the RAPID Cycle 6 pools over the SELEX pools. As mentioned previously, the top aptamer candidates from these two methods were actually resolved by Cycle 4 using both methods. This reflects the power of high-throughput sequencing for identifying enriching aptamers with great sensitivity many cycles before true convergence. In this case, the candidates from SELEX Cycle 4 represented only 1 part in 10⁵ sequences. However, these same candidates were about 10-fold more enriched after four cycles (two rounds) of RAPID, at about 1 part in 10⁴, which would increase our confidence that these were true aptamer candidates had we stop after only four cycles. For many applications, a great deal of effort is dedicated to isolating and perfecting an aptamer candidate into an ideal diagnostic or therapeutic reagent with particular characteristics. However, the emphasis of this work is the description of a novel selection method, RAPID, and its comparison to conventional SELEX. The development and characterization of CHK2 and UBLCP1 specific aptamers is beyond the scope of this work and therefore not fully investigated. However, we isolated our best candidate aptamer for CHK2, C6M1, and show that the raw aptamer was indeed able to bind to its target. Although both methods were effective at enriching many common sequences to each target, the RAPID method performed with sub-optimal parameters was able to generate the same results in only one third the time as SELEX performed under optimal conditions.

In addition to the specific protein binding results, we were also able to study the impact of Empty microcolumns and downstream processing may have had on the selections. Interestingly, we noticed that the Empty microcolumn generally bound a comparable amount of RNA as the two protein targets (FIG. 2). This is not surprising because true aptamers with high affinity and specificity are assumed to be rare in the starting library; nearly all the recovered sequences in any initial selection represent background and non-specific binding sequences. Despite the comparable binding observed within the Empty microcolumns, there was minimal multiplicity evolution from cycle to cycle (FIG. 3). This resulted in similar multiplicity distributions across the Empty microcolumn selected pools. The collective set of high-throughput sequencing results for the Empty microcolumns suggest that there was negligible sequence bias in the starting random pool (Cho et al. 2010) as well as negligible contributions from the microcolumns and the enzymatic processes (PCR, transcription, etc.) to the overall sequence enrichment in the two protein target pools (Zimmermann et al. 2010). We also found that for the Empty microcolumn pools there was no relationship between sequence multiplicity and enrichment (FIG. 4) since no specific binding and elution was taking place. High ranking sequences in any Empty target cycle are most likely to be random due to the nature of non-specific binding and the specific elutions used, so sequences identified in one cycle should be uncorrelated with those in the next cycle. In contrast, for our protein target selections, target-bound aptamers were eluted specifically together with the protein by disrupting the hexahistidine-tag and Ni⁺²-NTA interaction by EDTA.

While we have chosen to perform RAPID using the simple pairing of one Non-Amplification Cycle followed by one Amplification Cycle, the efficiency of RAPID may be further improved through alternative configurations. In general, more Non-Amplification Cycles can be performed between Amplification cycles, though the number will be limited by practical considerations. In the initial cycles, care should be taken to ensure that for large diverse libraries with low copy numbers, the stochastics of binding do not result in the complete loss of too many real aptamers, especially when total binding is very low. When this is a concern, amplification can be done to restore aptamers to sufficient copy numbers. Conversely, in later cycles where affinity increases and the input concentration drops between non-amplification cycles, the fractional recovery and total binding will increase (FIG. 2B), diminishing the yield for continued efforts. Once again, amplification can be done to increase the total concentration and to promote more competitive binding. Amplification is an essential part of the selection process when identifying candidates using population-based metrics. However its application should be minimized within the constraints listed above in order to optimize selection strategies for time rather than for individual cycle performances. However, our results make a compelling case for RAPID both in its time efficiency, and its cycle-to-cycle performance.

To summarize, we developed a new generalized method, RAPID, to rapidly select RNA aptamers. Our analyses show that even with only half the amplification cycles as SELEX, RAPID improves the overall selection performance. RAPID generated more enriched sequence distributions than did SELEX at an equivalent number of cycles, while enriching a significant fraction of identical sequences as SELEX for both protein targets. This demonstrates that RAPID is capable of efficiencies far greater than SELEX and that the reduced input materials and concentrations used in RAPID may prove beneficial in selections. Further improvements from alternative configurations of RAPID need to be investigated in the future. Although we used our microcolumn-based processes to perform all selections, our RAPID method may be used in combination with any selection mode or technology to save time, reagents, and to rapidly converge selection pools. This may be particularly useful for difficult selections requiring many cycles, or when complete sequence convergence is needed so that conventional cloning methods can be used to identify candidates. Although the time-saving benefits would be less compared to RNA-based selections, RAPID can also be extended to DNA or other modified nucleic acid selections in order to reduce costs and to improve selections for high affinity aptamers. We used high-throughput sequencing to quantify selected pools as described by histograms of evolving multiplicities, and scatter plots of sequence enrichments and identical sequences derived from two independent selection methods. Similar detailed analyses could be used to gain higher confidence in aptamer candidates through replicate selections, or to make more quantitative evaluations of different selection schemes and technologies. In particular, with a standardized pool and target, these analyses could be used to objectively rank and compare different selection techniques.

### Example 2

### RAPID Versus SELEX

Experiments showing RAPID performance over SELEX performance were performed with two different libraries and a number of targets. Two of these have been studied in great detail: NELF-E, and a domain of NELF-E, the RNA Recognition Motif referred to as the NELF-E RRM. The data is summarized in Table 1. From these data, a high affinity aptamer motif has been identified that associates with both of these related targets.

TABLE 1
Comparison of RAPID and SELEX for
NELF-E and NELF-E RRM targets

| | | | | APTAMER | |
|---|---|---|---|---|---|
| | | | | MOTIF | EST. % |
| | LI- | | CYCLE/ | FRE- | OF |
| TARGET | BRARY | METHOD | ROUND | QUENCY | POOL |
| NELF-E | N70 | SELEX | 4/4 | 0/300 | <7 × 10⁻⁵ |
| | | | 6/6 | 148/300 | ~0.6 |
| | N70 | RAPID | 4/2 | 0/300 | <0.009 |
| | | | 6/3 | 30/300 | ~1.4 |
| | GRO- | RAPID | 4/2 | 226/900 | <25 |
| | RNA | | | | |
| NELF-E | N70 | SELEX | 4/4 | 0/300 | <0.005 |
| RRM | | | 6/6 | 205/300 | ~41 |
| | N70 | RAPID | 4/2 | 129/300 | ~14 |
| | | | 6/3 | 168/300 | ~53 |

For NELF-E the relevant aptamer sequence was not detected at an enriched level after 4 cycles (4 rounds) of SELEX or in 4 cycles (2 rounds) of RAPID. However the RAPID Cycle 4 pool was more evolved than the SELEX Cycle 4 pool where the top 300 sequences in RAPID represented a 10-fold higher fraction of the entire pool than SELEX. After 6 Cycles of SELEX (6 rounds), the relevant motif was found in 148 sequences out of 300 (49%), which is estimated to be present at 0.6% of the entire pools contents. However, for RAPID, after 6 Cycles of SELEX (3 rounds), the motif was found in 30 of the top 300 sequences (10%). Although this frequency is lower, the pool is much further evolved than in SELEX and the motif is estimated to represent 1.4% of the entire pool's contents. This is 2-3 fold more enriched in RAPID than in SELEX which is consistent with our previous results, where RAPID took only half the time/reagents to complete as SELEX.

An additional RAPID selection experiment was performed against NELF-E with a genomic Global Run-On (GRO) RNA library to investigate the biological significance of the RNA aptamer motif Interestingly, after only 4 Cycles of RAPID (2 Rounds), the motif was identified in 226 of the top 900 sequences (25%). This selection took only one third the time and reagents to achieve similar results via SELEX.

For the NELF-E RRM the sequence motif was not identified as enriched in the top 300 sequences after 4 Cycles (4 rounds) of SELEX. However, after 4 Cycles of RAPID (2 rounds), the motif was detected 129 times in the top 300 sequences (43%) and is estimated to already represent 14% of the entire pool's contents. The motif was detected in SELEX after 6 Cycles (6 rounds) in 205 of the top 300 sequences, which is estimated to represent 41% of the entire pool's contents. After 6 Cycles (3 rounds) of RAPID, the motif was identified in 168 of the top 300 sequences. Although this frequency is slightly lower than in SELEX, it is estimated to represent 53% of the entire pool's contents. Not only is this still more enriched than in SELEX, but the motif had already nearly converged after only 4 Cycles of RAPID, where it was undetectable after the same number of Cycles in SELEX. This resulted in comparable performance between 6 Cycles of SELEX and 4 Cycles of RAPID, where RAPID required only one third the time/reagents as SELEX.

### Example 3

### RNA Binding Experiments

Table 2 is a summary of the binding to various targets given as the amount of molecules in pico-moles. The amount given is the number of molecules entering each cycle (i.e. Cycle 1, Cycle 2), or the number of molecules remaining after each Round before amplification (i.e. Post ½).

TABLE 2
RNA Binding

| | | | Post | | | Post | | | Post |
|---|---|---|---|---|---|---|---|---|---|
| | Cycle 1 | Cycle 2 | 1/2 | Cycle 3 | Cycle 4 | 3/4 | Cycle 5 | Cycle 6 | 5/6 |
| TARGET | pmol | pmol | pmol | pmol | pmol | pmol | pmol | pmol | pmol |
| Empty | 40111 | 3.052 | 0.0176 | 2006 | 0.220 | 0.0083 | 100 | 0.04 | 0.0112 |
| CHK2 | 40111 | 2.454 | 0.0058 | 2006 | 0.247 | 0.0034 | 100 | 0.15 | 0.0236 |
| UBLCP1 | 40111 | 2.598 | 0.0037 | 2006 | 0.326 | 0.0023 | 100 | 0.06 | 0.0044 |
| Average | 40111 | 2.680 | 0.009 | 2006 | 0.235 | 0.0047 | 100 | 0.08 | 0.0131 |

Calculated odds of being sampled from binding

| | | | | | | |
|---|---|---|---|---|---|---|
| Binding | 1:14286 | 1:298 | 1:8333 | 1:50 | 1:1186 | 1:6 |

Copy number Amplification factor

| | | | |
|---|---|---|---|
| Amplify | 5x | 222889x | 21413x |

As mentioned herein, it is generally true that all observed binding is in fact background binding, as any one aptamer represents a negligible fraction of the pool (this is true until convergence when the pool binding becomes dominated by the aptamer). This being the case, we expect to see similar binding from the Empty columns as with the Target proteins which is what we observe. For this reason the average binding behavior is also calculated and assumed to be representative of all targets, and this is used to calculate the probability of any molecule being bound at each cycle of selection. Also included in Table 2 is the amplification factor “X” going into the next round (i.e., after amplification steps), which is to say that on average, the copy numbers are “X” times larger, but the fractions of any sequence in the pool remain the same.

### Example 4

### Guiding Rules for Amplification/Non-Amplification Cycles

Given the summary of RNA binding, Table 3 generalizes the average background binding behavior (BKG) as the probability for being sampled, to the nearest order of magnitude i.e. for Cycle 1, 1 in 10⁴ molecules were sampled.

TABLE 3
Rules for Amplification/Non-Amplification Cycles

| | | | Post | | | Post | | | Post |
|---|---|---|---|---|---|---|---|---|---|
| SPECIES | Cycle 1 | Cycle 2 | 1/2 | Cycle 3 | Cycle 4 | 3/4 | Cycle 5 | Cycle 6 | 5/6 |
| | Binding Odds | | | | | | | | |
| BKG | 1:10⁴ | 1:10² | | 1:10⁴ | 1:10² | | 1:10³ | 1:10¹ | |
| APT | 1:10 | 1:10 | | 1:10 | 1:10 | | 1:10 | 1:10 | |
| | Hypothetical Aptamer Numbers | | | | | | | | |
| Apt # | 100 | 10 | 1 | 222889 | 22289 | 2229 | 47729577 | 4772958 | 477296 |
| Apt pmol | 1 × 10⁻¹⁰ | 1 × 10⁻¹¹ | 1 × 10⁻¹² | 4 × 10⁻⁷ | 4 × 10⁻⁸ | 4 × 10⁻⁹ | 8 × 10⁻⁵ | 8 × 10⁻⁶ | 8 × 10⁻⁷ |

Very efficient recoveries of aptamers when doped into full size libraries have been demonstrated (e.g., GFP experiments in Latulippe et al. 2013). If some conservative estimates are made, one can determine the boundary conditions for the two modes of selection. Assume that sequences with high affinity to a target binds no less than 10% of the time, which is a conservatively low estimate, and that there are at least a 100 of these molecules (which is very conservative since many believe they can be as abundant as 1 in a million). Using actual data for background binding above, the minimum expected number for an “aptamer” of interest in the pool given the stated assumptions has been determined.

Here, one can see that in the illustrative example, one expects to recover only 1 high affinity molecule after 2 cycles of selections, while the background binding was very low, i.e., less than the probability of binding an aptamer. This one molecule is then amplified 222889-fold going into cycle 3. Once again, it is found that the background binding was still much lower than the minimum probability for binding an aptamer for both Cycle 3 and 4. However, this time, after cycle 4, there were still over 1000 copies of the aptamer. Although amplification was performed here, more non-amplification cycles likely could have been performed.

According to the assumed numbers, at least 2-3 more cycles could have been performed while being sure that the aptamer was still present in the pool. Going into Cycle 5, the aptamer underwent a 21413-fold amplification and after two more cycles, there were about half a billion copies remaining in the pool. In this case, it is found that, although many more cycles (at least 5) could have been performed and that one could still be certain the aptamer was still present in the pool, the background binding had risen to about 10%, which is the assumed (minimum) binding probability of the aptamer. This behavior would suggest that either the aptamer had converged (though it had not), or that the background binding was too high because the library concentration was too low. Either way one could then perform amplification, and work at much higher concentrations of molecules. However, at this point, one can calculate that the aptamer should be present at least 1 in 10⁴-10⁵, which is detectable with sequencing methods, and may not require any more selections steps in order to identify aptamer candidates. In actuality, the aptamers identified from the actual data were present at about 1 in 1000, so this example is accurate and underestimates the actual binding probability.

Therefore, in various embodiments, one could start with at least 1 assumption: the probability of binding an aptamer molecule, P(A) (and possibly the number of such molecules you believe to be present, N(A); which must always be greater than or equal to 1). Using this assumed probability P(A), and the measured probability of binding background molecules P(B,n,i) at the n^{th} cycle, one should perform amplifications once the expected copy number of aptamer molecules falls below some acceptable threshold Nₘᵢₙ (e.g., 10 molecules), or when the measured background binding probability approaches the assumed aptamer binding probability within some minimum acceptable enrichment factor Eₘᵢₙ (e.g., within 1/10 of P(A)), i.e., the number of Cycles ‘i’ before amplification are constrained such that:

Nₘᵢₙ×P(A)≧N(A)×P(A)ⁱ; where Nₘᵢₙ×P(A)≧1; i.e. Nₘᵢₙ≧N(A)×P(A)ⁱ⁻¹ where Nₘᵢₙ≧P(A)⁻¹≧1 and i≧1 (1)

Eₘᵢₙ≧P(A)/P(B,n,i); where Eₘᵢₙ>1 and n≧i≧1 (2)

where:
N(A)=number of such molecules believed to be present, where N(A)≧1;
P(A)=probability of binding an aptamer molecule;
P(B,n,i)=measured probability of binding background molecules at the nth cycle with i cycles performed after the last amplification; and
i=the number of selection cycles before an amplification cycle is to be performed.

Once either of the two above inequalities becomes untrue after “i” cycles, then amplification needs to be done or should be done. This can result in values that change after every amplification cycle. If one looks at the data provided, it appears that in general an amplification was performed once there was <0.01 pico-mols of RNA left. This can then be amplified up to arbitrarily high numbers so as to achieve a higher possible number of cycles before another amplification “i”.

In another embodiment, a more constrained approach can be taken. This is accomplished by simply constraining the parameter “i” such that one must always skip at least 1 amplification step (i.e., i>1), and that one must perform at least one amplification step (i<M, where M is the total number of selection cycles to be performed). Using this approach the guiding formulae or equations can be as follows:

Nₘᵢₙ×P(A)≧N(A)×P(A)ⁱ; where Nₘᵢₙ×P(A)≧1; i.e. Nₘᵢₙ≧N(A)×P(A)ⁱ⁻¹ where Nₘᵢₙ≧P(A)⁻¹≧1 and M>i>1 (1)

Eₘᵢₙ≧P(A)/P(B,n,i); where Eₘᵢₙ≧1 and n≧i≧1, and M>i (2)

where:
M=total number of selection cycles to be performed;
N(A)=number of such molecules believed to be present, where N(A)≧1;
P(A)=probability of binding an aptamer molecule;
P(B,n,i)=measured probability of binding background molecules at the nth cycle with i cycles performed after the last amplification; and
i=the number of selection cycles before an amplification cycle is to be performed.

### REFERENCES

Citation of a reference herein shall not be construed as an admission that such reference is prior art to the present invention. All references cited herein are hereby incorporated by reference in their entirety. Certain references are cited herein by author and date. Below is a listing of various references cited herein, with the references being identified by author, date, publication, and page numbers:
J. Ashley et al., (2012) Electrophoresis 33(17): 2783-2789.
M. Berezovski et al., (2006) Journal of the American Chemical Society 128(5): 1410-1411.
P. E. Burmeister et al., (2005) Chemistry & biology 12(1): 25-33.
M. Cho et al., (2010) Proceedings of the National Academy of Sciences of the United States of America 107(35): 15373-15378.
J. Ciesiolka et al., (1995) Rna 1(5): 538-550.
J. C. Cox et al., (2001) Bioorganic & medicinal chemistry 9(10): 2525-2531.
J. C. Cox et al., (1998) Biotechnology progress 14(6): 845-850.
D. A. Daniels et al., (2003) Proceedings of the National Academy of Sciences of the United States of America 100(26): 15416-15421.
A. D. Ellington et al., (1990) Nature 346(6287): 818-822.
A. Geiger et al., (1996) Nucleic Acids Res 24(6): 1029-1036.
J. Gevertz et al., (2005) Rna 11(6): 853-863.
L. Gold et al., (2010) PloS one 5(12): e15004.
Q. Gong et al., (2012) Analytical chemistry 84(12): 5365-5371.
L. S. Green et al., (1995) Chemistry & biology 2(10): 683-695.
R. D. Jenison et al., (1994) Science 263(5152): 1425-1429.
K. B. Jensen et al., (1995) Proceedings of the National Academy of Sciences of the United States of America 92(26): 12220-12224.
A. Jolma et al., (2010) Genome research 20(6): 861-873.
G. F. Joyce (1989) Gene 82(1): 83-87.
S. Klussmann et al., (1996) Nat Biotechnol 14(9): 1112-1115.
J. A. Latham et al., (1994) Nucleic Acids Res 22(14): 2817-2822.
D. R. Latulippe et al., (2013) Analytical chemistry.
S. D. Mendonsa et al., (2004) Analytical chemistry 76(18): 5387-5392.
A. Nitsche et al., (2007) Bmc Biotechnol 7.
A. Ozer et al., (2013) Nucleic Acids Res.
J. M Pagano et al., (2007) The Journal of biological chemistry 282(12): 8883-8894.
J. S. Paige et al., (2011) Science 333(6042): 642-646.
S. M. Park et al., (2009) Lab on a chip 9(9): 1206-1212.
L. Peng et al., (2007) Microsc Res Techniq 70(4): 372-381.
M. S. L. Raddatz et al., (2008) Angew Chem Int Edit 47(28): 5190-5193.
J. Ruckman et al., (1998) The Journal of biological chemistry 273(32): 20556-20567.
T. Schutze et al., (2011) PloS one 6(12): e29604.
W. H. Thiel et al., (2011) Nucleic Acid Ther 21(4): 253-263.
J. Tok et al., (2010) Electrophoresis 31(12): 2055-2062.
C. Tuerk et al., (1990) Science 249(4968): 505-510.
B. Zimmermann et al., (2010) PloS one 5(2): e9169.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method for selecting an aptamer for a target molecule, said method comprising:
providing a random oligonucleotide library comprising a plurality of unique random sequence oligonucleotides;
providing a target mixture comprising at least one target molecule; and
subjecting the random oligonucleotide library and the target mixture to at least one round of an aptamer isolation protocol to yield at least one aptamer for the target molecule, wherein a round of the aptamer isolation protocol comprises at least one selection cycle followed by an amplification cycle,
wherein said at least one selection cycle comprises: (i) contacting the random oligonucleotide library with the target mixture to bind oligonucleotides to the target molecule; and (ii) isolating the bound oligonucleotides to yield an enriched oligonucleotide pool comprising a plurality of high affinity oligonucleotides that bind with specificity to the target molecule; and
wherein said amplification cycle comprises subjecting the enriched oligonucleotide pool to an amplification process to yield an amplified oligonucleotide pool comprising an increased number of copies of the plurality of high affinity oligonucleotides.

2. The method according to claim 1 further comprising:
determining that an amplification cycle trigger point has been reached before performing the amplification cycle,
wherein the amplification cycle trigger point is reached when either of the following occurs:
(a) aptamer molecule numbers fall below a minimum acceptable number of molecules (Nₘᵢₙ); or
(b) measured background binding probability approaches an assumed binding probability within a minimum acceptable enrichment factor (Eₘᵢₙ).

3. The method according to claim 2, wherein the number of selection cycles (denoted as “i”) before an amplification cycle is to be performed is determined based on the minimum acceptable number of molecules (Nₘᵢₙ) as calculated according to Formula I as follows:
Nₘᵢₙ×P(A)≦N(A)×P(A)ⁱ (Formula I)
wherein:
Nₘᵢₙ×P(A)≧1;
Nₘᵢₙ≦N(A)×P(A)ⁱ⁻¹, where Nₘᵢₙ≧P(A)⁻¹≧1 and i≧1;
N(A)=number of such molecules believed to be present, where N(A)≧1;
P(A)=probability of binding an aptamer molecule; and
i=the number of selection cycles before an amplification cycle is to be performed, and
wherein the amplification cycle trigger point is reached and an amplification cycle is to be performed once the inequality of Formula I becomes untrue after “i” cycles.

4. The method according to claim 2, wherein the number of selection cycles (denoted as “i”) before an amplification cycle is to be performed is determined based on the minimum acceptable number of molecules (Nₘᵢₙ) as calculated according to Formula I as follows:
Nₘᵢₙ×P(A)≦N(A)×P(A)ⁱ (Formula I)
wherein:
Nₘᵢₙ×P(A)≧1;
Nₘᵢₙ≦N(A)×P(A)ⁱ⁻¹, where Nₘᵢₙ≧P(A)⁻¹≧1 and M>i>1;
M=total number of selection cycles to be performed;
N(A)=number of such molecules believed to be present, where N(A)≧1;
P(A)=probability of binding an aptamer molecule; and
i=the number of selection cycles before an amplification cycle is to be performed, and
wherein the amplification cycle trigger point is reached and an amplification cycle is to be performed once the inequality of Formula I becomes untrue after “i” cycles.

5. The method according to claim 2, wherein the number of selection cycles (denoted as “i”) before an amplification cycle is to be performed is determined based on the minimum acceptable enrichment factor (Eₘᵢₙ) as calculated according to Formula II as follows:
Eₘᵢₙ≦P(A)/P(B,n,i) (Formula II)
wherein:
Eₘᵢₙ>1 and n≧i≧1;
P(A)=probability of binding an aptamer molecule;
P(B,n,i)=measured probability of binding background molecules at the n^{th} cycle with i cycles performed after the last amplification; and
i=the number of selection cycles before an amplification cycle is to be performed,
wherein the amplification cycle trigger point is reached and an amplification cycle is to be performed once the inequality of Formula II becomes untrue after “i” cycles.

6. The method according to claim 2, wherein the number of selection cycles (denoted as “i”) before an amplification cycle is to be performed is determined based on the minimum acceptable enrichment factor (Eₘᵢₙ) as calculated according to Formula II as follows:
Eₘᵢₙ≦P(A)/P(B,n,i) (Formula II)
wherein:
Eₘᵢₙ>1 and n≧i≧1 and M>i;
P(A)=probability of binding an aptamer molecule;
P(B,n,i)=measured probability of binding background molecules at the n^{th} cycle with i cycles performed after the last amplification; and
i=the number of selection cycles before an amplification cycle is to be performed,
wherein the amplification cycle trigger point is reached and an amplification cycle is to be performed once the inequality of Formula II becomes untrue after “i” cycles.

7. The method according to claim 2, wherein the determining is performed after one selection cycle, after two selection cycles, after three selection cycles, or after more than three selection cycles.

8. The method according to claim 2, wherein the Nₘᵢₙ has a value in a range selected from the group consisting of from between about 1 and about 500 aptamer molecules, between about 1 and about 400 aptamer molecules, between about 1 and about 300 aptamer molecules, between about 1 and about 200 aptamer molecules, between about 1 and about 100 aptamer molecules, between about 1 and about 90 aptamer molecules, between about 1 and about 80 aptamer molecules, between about 1 and about 70 aptamer molecules, between about 1 and about 60 aptamer molecules, between about 1 and about 50 aptamer molecules, between about 1 and about 40 aptamer molecules, between about 1 and about 30 aptamer molecules, between about 1 and about 20 aptamer molecules, between about 1 and about 15 aptamer molecules, between about 1 and about 10 aptamer molecules, and between about 1 and about 5 aptamer molecules.

9. The method according to claim 2, wherein the Eₘᵢₙ has a value in a range selected from the group consisting of within about 1/1000 of probability of binding an aptamer molecule (denoted as “P(A)”), within about 1/500 of P(A), within about 1/400 of P(A), within about 1/300 of P(A), within about 1/200 of P(A), within about 1/100 of P(A), within about 1/50 of P(A), within about 1/25 of P(A), within about 1/20 of P(A), within about 1/15 of P(A), within about 1/10 of P(A), within about ⅕ of P(A), within about 1 of P(A), and within about 10 of P(A).

10. The method according to claim 1, wherein the number of selection cycles to be performed in a particular round is dependent on reaching an amplification cycle trigger point,
wherein the amplification cycle trigger point is reached when either of the following occurs:
(a) aptamer molecule numbers fall below a minimum acceptable number of molecules (Nₘᵢₙ); or
(b) measured background binding probability approaches an assumed binding probability within a minimum acceptable enrichment factor (Eₘᵢₙ).

11. The method according to claim 1, wherein the random oligonucleotide library and the target mixture are subjected to one round, two rounds, three rounds, or more than three rounds of the aptamer isolation protocol.

12. The method according to claim 1, wherein one round of the aptamer isolation protocol is selected from the group consisting of one selection cycle followed by one amplification cycle, two selection cycles followed by one amplification cycle, three selection cycles followed by one amplification cycle, four selection cycles followed by one amplification cycle, and more than four selection cycles followed by one amplification cycle.

13. The method according to claim 1, wherein the amplification cycle is performed once there is about <0.10 pico-mols of oligonucleotides, about <0.05 pico-mols of oligonucleotides, about <0.04 pico-mols of oligonucleotides, about <0.03 pico-mols of oligonucleotides, about <0.02 pico-mols of oligonucleotides, or about <0.01 pico-mols of oligonucleotides left in the enriched oligonucleotide pool.

14. The method according to claim 1, wherein the random oligonucleotide library is a random RNA oligonucleotide library or a random DNA oligonucleotide library.

15. The method according to claim 1, wherein the aptamer is selected from the group consisting of an RNA aptamer and a DNA aptamer.

16. The method according to claim 1, wherein the target molecule is selected from the group consisting of a whole cell, a virus, a protein, a modified protein, a polypeptide, a modified polypeptide, an RNA molecule, a DNA molecule, a modified DNA molecule, a polysaccharide, an amino acid, an antibiotic, a pharmaceutical agent, an organic non-pharmaceutical agent, a macromolecular complex, a carbohydrate, a small molecule, a chemical compound, a mixture of lysed cells, and a mixture of purified, partially purified, or non-purified protein.

17. The method according to claim 1, wherein isolating the bound oligonucleotides to yield the enriched oligonucleotide pool comprises:
washing unbound and weakly bound oligonucleotides from the target mixture; and
eluting the oligonucleotides that specifically bind to the target molecules, wherein the eluted oligonucleotides are aptamers that bind to the target molecules.

18. The method according to claim 17, wherein when the oligonucleotide aptamers comprise RNA aptamers, the method further comprises:
performing reverse transcription amplification of the selected aptamer population.

19. The method according to claim 18 further comprising:
purifying and sequencing the amplified apatmer population.

20. The method according to claim 19, wherein said isolating, said performing reverse transcription amplification, said purifying, and/or said sequencing are performed in one or more separate fluidic devices coupled in fluidic communication with a microcolumn device suitable for maintaining a target molecule.

21. A method for selecting an aptamer for a target molecule, said method comprising:
providing a random oligonucleotide library comprising a plurality of unique random sequence oligonucleotides;
providing a target mixture comprising at least one target molecule; and
subjecting the random oligonucleotide library and the target mixture to multiple rounds of an aptamer isolation protocol to yield at least one aptamer that binds with specificity and high affinity to the target molecule,
wherein one round of an aptamer isolation protocol comprises multiple non-amplification selection cycles followed by one amplification cycle,
wherein said multiple non-amplification selection cycles initially comprises:
(i) contacting the random oligonucleotide library with the target mixture to selectively bind a fraction of the oligonucleotide library to the target molecule;
(ii) isolating the bound oligonucleotides to yield an enriched oligonucleotide pool;
(iii) contacting the enriched oligonucleotide pool with the target mixture to selectively bind a fraction of the oligonucleotide pool to the target molecule; and
(iv) repeating steps (ii) and (iii) to obtain an amount of the enriched oligonucleotide pool comprising a plurality of high affinity oligonucleotides, remaining for the amplification cycle,
wherein said amplification cycle comprises subjecting the enriched oligonucleotide pool to an amplification process to yield an amplified oligonucleotide pool comprising an increased number of copies of the plurality of high affinity oligonucleotides.

22. The method according to claim 21, wherein the multiple non-amplification selection cycles comprises two selection cycles, three selection cycles, or more than three selection cycles.

23. The method according to claim 21, wherein the random oligonucleotide library and the target mixture are subjected to two rounds, three rounds, or more than three rounds of the aptamer isolation protocol.

24. The method according to claim 21, wherein one round of the aptamer isolation protocol is selected from the group consisting of two selection cycles followed by one amplification cycle, three selection cycles followed by one amplification cycle, four selection cycles followed by one amplification cycle, and more than four selection cycles followed by one amplification cycle.

25. The method according to claim 21, wherein the amplification cycle is performed once there is about <0.10 pico-mols of oligonucleotides, about <0.05 pico-mols of oligonucleotides, about <0.04 pico-mols of oligonucleotides, about <0.03 pico-mols of oligonucleotides, about <0.02 pico-mols of oligonucleotides, or about <0.01 pico-mols of oligonucleotides left in the enriched oligonucleotide pool.

26. The method according to claim 21, wherein the random oligonucleotide library is a random RNA oligonucleotide library or a random DNA oligonucleotide library.

27. The method according to claim 21, wherein the aptamer is selected from the group consisting of an RNA aptamer and a DNA aptamer.

28. The method according to claim 21, wherein the target molecule is selected from the group consisting of a whole cell, a virus, a protein, a modified protein, a polypeptide, a modified polypeptide, an RNA molecule, a DNA molecule, a modified DNA molecule, a polysaccharide, an amino acid, an antibiotic, a pharmaceutical agent, an organic non-pharmaceutical agent, a macromolecular complex, a carbohydrate, a small molecule, a chemical compound, a mixture of lysed cells, and a mixture of purified, partially purified, or non-purified protein.


SEQUENCE LISTING (24 sequences)

SEQ ID NO: 1 - DNA, 50 bp, Artificial Sequence
  DNA Aptamer
taataataaaagagtgaccatcccaaataacgtcaatatacgcaatacgg

SEQ ID NO: 2 - DNA, 50 bp, Artificial Sequence
  DNA Aptamer
aaatatagaataccgcatagtattcctaaatatacgtcagattgatccat

SEQ ID NO: 3 - DNA, 50 bp, Artificial Sequence
  DNA Aptamer
taataataaagggtggctattcccagcttcctcttgactcgcaccgtaaa

SEQ ID NO: 4 - DNA, 50 bp, Artificial Sequence
  DNA Aptamer
gttctagctgcagatacaactgagccaaataaactatcgtagtacgttgc

SEQ ID NO: 5 - DNA, 50 bp, Artificial Sequence
  DNA Aptamer
caaaatatacagatcctcgcaatagggggcctgacttcaaatagggaatc

SEQ ID NO: 6 - DNA, 50 bp, Artificial Sequence; identical to SEQ ID NO: 3
  DNA Aptamer

SEQ ID NO: 7 - DNA, 50 bp, Artificial Sequence
  DNA Aptamer
caagaataaaggacggaagcatttccgagcgactacatcaagtcggaaca

SEQ ID NO: 8 - DNA, 50 bp, Artificial Sequence
  DNA Aptamer
taataataaagggtgtttccagcccgacttaagctccgcgactaaccaac

SEQ ID NO: 9 - DNA, 50 bp, Artificial Sequence
  DNA Aptamer
taagaataaaagagtcgatgcacttccacacttagcttcaaccggcgaat

SEQ ID NO: 10 - DNA, 50 bp, Artificial Sequence; identical to SEQ ID NO: 1
  DNA Aptamer

SEQ ID NO: 11 - DNA, 50 bp, Artificial Sequence
  DNA Aptamer
gatcggttccaacgctctgtcgcctaagtgaacagatgaagaaaaaatag

SEQ ID NO: 12 - DNA, 50 bp, Artificial Sequence
  DNA Aptamer
atgacctcgtcatcacccgtatgagcagcccaaagacaaaaattcgcgag

SEQ ID NO: 13 - DNA, 50 bp, Artificial Sequence
  DNA Aptamer
atgaccagttcaccttaggtctacggctaggaatctaggattaaataata

SEQ ID NO: 14 - DNA, 50 bp, Artificial Sequence
  DNA Aptamer
caagccttcactccgcaaaatacgcgcttccaaataataacaataagaac

SEQ ID NO: 15 - DNA, 50 bp, Artificial Sequence
  DNA Aptamer
cagtcctagcacaagactgcaacgtggatatgaccccatactgactaaca

SEQ ID NO: 16 - DNA, 50 bp, Artificial Sequence; identical to SEQ ID NO: 11
  DNA Aptamer

SEQ ID NO: 17 - DNA, 50 bp, Artificial Sequence; identical to SEQ ID NO: 14
  DNA Aptamer

SEQ ID NO: 18 - DNA, 50 bp, Artificial Sequence
  DNA Aptamer
caacgagatacaataataacaataaccaccccgattcgttccgtgatcca

SEQ ID NO: 19 - DNA, 50 bp, Artificial Sequence
  DNA Aptamer
acaagctttatcctaacagccagagacctcaccacccagagaagagcaga

SEQ ID NO: 20 - DNA, 50 bp, Artificial Sequence
  DNA Aptamer
atacacttcatcaccccagagctgtgttgctgaaaggcctacctacagag

SEQ ID NO: 21 - DNA, 71 bp, Artificial Sequence
  DNA Aptamer
gatcggttccaacgctctgtcgcctaagtgaacagatgaagaaaaaatagcccaataagaggcaacaatct

SEQ ID NO: 22 - DNA, 120 bp, Artificial Sequence
  DNA Aptamer
aagcttcgtcaagtctgcagtgaannnnnnnnnnnnnnnnnnnnnnnnnnnnnnnnnnnnnnnnnnnnnnnnnnnnnnnnnnnnnnnnnnnnnngaattcgtagatgtggatccattccc

SEQ ID NO: 23 - DNA, 41 bp, Artificial Sequence
  Lib-FOR Oligonucleotide
gataatacgactcactatagggaatggatccacatctacga

SEQ ID NO: 24 - DNA, 24 bp, Artificial Sequence
  Lib-REV oligonucleotide
aagcttcgtcaagtctgcagtgaa